(19) United States
(12) Patent Application Publication
(10) Pub. No.: US 2026/0139229 A1
(43) Pub. Date: May 21, 2026

(54) MULTILINEAGE CARDIOVASCULAR ORGANOIDS AND METHODS OF GENERATING THE SAME

(71) Applicant: The University of Chicago, Chicago, IL (US)

(72) Inventors: Yoav Gilad, Chicago, IL (US); Erik M. McIntire, Chicago, IL (US); Katherine L. Rhodes, Chicago, IL (US)

(21) Appl. No.: 19/118,407

(22) PCT Filed: Oct. 4, 2023
(86) PCT No.: PCT/US2023/034422

§ 371 Date: Apr. 4, 2025

Related U.S. Application Data
(60) Provisional application No. 63/413,358, filed on Oct. 5, 2022.

(51) Int. Cl. C12N 5/071; C12Q 1/6876

(52) U.S. Cl. CPC C12N 5/0697; C12Q 1/6876; C12N 2501/415; C12N 2503/04; C12N 2506/45; C12Q 2600/142; C12Q 2600/158

(57) ABSTRACT

Provided herein are multilineage cardiovascular organoids and methods of generating the same. In some aspects, provided herein is a system comprising a plurality of multilineage cardiovascular organoids derived from embryoid bodies. The embryoid bodies can be aggregated from pluripotent stem cells of varying genotypes. The multilineage cardiovascular organoids and systems described herein may be used for screening of agents, such as anti-cancer agents, for cardiotoxicity.

### PRIORITY STATEMENT

This application claims priority to U.S. Provisional Ser. No. 63/413,358, filed Oct. 5, 2022, the entire contents of which are incorporated herein by reference.

### STATEMENT REGARDING FEDERAL FUNDING

This invention was made with government support under CA014599, and GM139782 awarded by the National Institutes of Health. The government has certain rights in the invention.

### BACKGROUND

Cardiovascular toxicity is a common and often debilitating side effect of various therapeutic drugs, including cancer therapies. For example, anticancer drug-induced cardiovascular toxicity (CT) encompasses a multitude of clinical manifestations, including arrhythmia, myocardial ischemia, pericarditis, hypertension, left ventricular dysfunction, and heart failure. Because its timing and presentation vary enormously between patients, often appearing years after cancer treatment has ceased, CT is often difficult for clinicians to prevent, detect, and monitor. Still, CT management is a crucial aspect of cancer treatment for many patients; for example, acute presentations of CT may lead physicians to lower drug dosage, which may improve heart health at the expense of reducing treatment efficacy. The ability to predict which patients are at high risk for drug-induced CT would allow clinicians to provide patients with safer and more effective treatment options. Moreover, effective models of cardiotoxicity would allow for an investigation of specific cardiac cell types affected by toxicity, and the specific mechanisms underlying drug-induced cardiotoxicity. Accordingly, what is needed are effective and practical models and methods for identification of risk factors and biomarkers for studying drug-induced cardiotoxicity, including anti-cancer drug-induced CT.

### BRIEF SUMMARY OF THE DISCLOSURE

In some aspects, provided herein are multilineage cardiovascular organoids comprising mesodermal and non-mesodermal cell types. In some embodiments, the mesodermal cell types include one or more cell types selected from cardiac progenitor cells, proliferating cells, cardiomyocytes, and fibroblasts. In some embodiments, the non-mesodermal cell types include endoderm epithelial cells, liver cells, intestinal cells, and/or cardiac neural crest cells.

In some embodiments, the multilineage cardiovascular organoid is used in a method of assessing cardiotoxicity of an agent or condition. In some embodiments, the method of assessing cardiotoxicity comprises contacting the multilineage cardiovascular organoid with the agent or subjecting the multilineage cardiovascular organoid to the condition, and evaluating a response to the agent or the condition in at least one cell type within the multilineage cardiovascular organoid. In some embodiments, evaluating a response to the agent or condition comprises measuring gene expression, activity, or regulation in the at least one cell type. Gene expression, activity, or regulation can be measured by a variety of suitable techniques.

Exemplary methods for evaluating gene expression, activity, or regulation include, but are not limited to, molecular analysis such as PCR-based methods or sequencing, measuring chromatin accessibility, or epigenetic analyses. In some embodiments, evaluating a response to the agent or the condition in the at least one cell type within the multilineage cardiovascular organoid comprises identifying one or more response expression quantitative loci (eQTLs) in the at least one cell type. In some embodiments, the one or more response eQTLs are identified using single-cell RNA sequencing (scRNA-seq).

In some aspects, provided herein are methods of generating a multilineage cardiovascular organoid. In some embodiments, the method of generating a multilineage cardiovascular organoid comprises culturing an embryoid body in a cardiac induction medium comprising a Wnt signaling activator; and culturing the embryoid body in a Wnt inhibitor medium comprising a Wnt signaling inhibitor. In some embodiments, the method further comprises culturing the embryoid body in a basal heart medium in between culturing the embryoid body in the cardiac induction medium and culturing the embryoid body in the Wnt inhibitor medium. In some embodiments, the embryoid body is generated from induced pluripotent stem cells (iPSCs).

In some embodiments, the method of generating a multilineage cardiovascular organoid comprises generating an embryoid body from induced pluripotent stem cells (iPSCs); and guiding differentiation of the embryoid body to a cardiac lineage. In some embodiments, guiding differentiation of the embryoid body to a cardiac lineage comprises culturing an embryoid body in a cardiac induction medium comprising a Wnt signaling activator. In some embodiments, guiding differentiation further comprises culturing the embryoid body in a Wnt inhibitor medium comprising a Wnt signaling inhibitor following culturing the embryoid body in the cardiac induction medium. In some embodiments, guiding differentiation further comprises culturing the embryoid body in a basal heart medium in between culturing the embryoid body in the cardiac induction medium and culturing the embryoid body in the Wnt inhibitor medium.

For any of the methods of generating a multilineage cardiovascular organoid described herein, the Wnt activator may be a small molecule GSK3 inhibitor. In some embodiments, the small molecule GSK 3 inhibitor is CHIR99021. In some embodiments, the cardiac induction medium comprises CHIR99021 at a concentration of about 4 μM. In some embodiments, the Wnt signaling inhibitor is a small molecule porcupine inhibitor. In some embodiments, the small molecule porcupine inhibitor is IWP-4, IWP-2, LGK974, C59, or ETC-159. In some embodiments, the Wnt inhibitor medium comprises IWP-4 at a concentration of about 2 μM.

In some embodiments, the method comprises culturing the embryoid body in a cardiac induction medium comprising a Wnt signaling activator for 12-48 hours, followed by culturing the embryoid body in a basal heart medium for 24-76 hours, followed by culturing the embryoid body in a Wnt inhibitor medium comprising a Wnt signaling inhibitor for 24-76 hours. In some embodiments, the method comprises culturing the embryoid body in a cardiac induction medium comprising the Wnt signaling activator CHIR99021 for 12-48 hours (e.g. 12-48 hours, 14-40 hours, 16-36 hours, 20-28 hours, 22-26 hours, 23-25 hours, or about 24 hours), followed by culturing the embryoid body in basal heart medium for 24-76 hours (e.g. 24-76 hours, 30-70 hours, 36-64 hours, 40-60 hours, 44-56 hours, 44-50 hours, 46-50 hours, 47-49 hours, or about 48 hours), followed by culturing the embryoid body in a Wnt inhibitor medium comprising a small molecule porcupine inhibitor for 24-76 hours (e.g. 24-76 hours, 30-70 hours, 36-64 hours, 40-60 hours, 44-56 hours, 44-50 hours, 46-50 hours, 47-49 hours, or about 48 hours). In some embodiments, the method comprises culturing the embryoid body in a cardiac induction medium comprising the Wnt signaling activator CHIR99021 at a concentration of about 1 μM-10 μM (e.g. about 1 μM, about 2 μM, about 3 μM, about 4 μM, about 5 μM, about 6 μM, about 7 μM, about 8 μM, about 9 μM, about 4 μM) for 12-48 hours (e.g. 12-48 hours, 14-40 hours, 16-36 hours, 20-28 hours, 22-26 hours, or about 24 hours), followed by culturing the embryoid body in basal heart medium for 24-76 hours (e.g. 24-76 hours, 30-70 hours, 36-64 hours, 40-60 hours, 44-56 hours, 44-50 hours, 46-50 hours, 47-49 hours, or about 48 hours), followed by culturing the embryoid body in a Wnt inhibitor medium comprising a small molecule porcupine inhibitor for 24-76 hours (e.g. 24-76 hours, 30-70 hours, 36-64 hours, 40-60 hours, 44-56 hours, 44-50 hours, 46-50 hours, 47-49 hours, or about 48 hours). In some embodiments, the Wnt inhibitor medium comprises the small molecule porcupine inhibitor (e.g. IWP-4, IWP-2, LGK974, C59, or ETC-159) at a concentration of about 0.5 μM to about 5 μM.

In some embodiments, the method comprises culturing the embryoid body in a cardiac induction medium comprising the Wnt signaling activator CHIR99021 at a concentration of about 1 μM-10 μM (e.g. about 1 μM, about 2 μM, about 3 μM, about 4 μM, about 5 μM, about 6 μM, about 7 μM, about 8 μM, about 9 μM, about 4 μM) for about 20-28 hours (e.g. about 20 hours, about 21 hours, about 22 hours, about 23 hours, about 24 hours, about 25 hours, about 26 hours, about 27 hours, about 28 hours) followed by culturing the embryoid body in basal heart medium for about 44-52 hours (e.g. about 44 hours, about 45 hours, about 46 hours, about 47 hours, about 48 hours, about 49 hours, about 50 hours, about 51 hours, about 52 hours) followed by culturing the embryoid body in a Wnt inhibitor medium comprising the small molecule porcupine inhibitor IWP-4 at a concentration of about 0.5 μM to about 5 μM for about 44-52 hours (e.g. about 44 hours, about 45 hours, about 46 hours, about 47 hours, about 48 hours, about 49 hours, about 50 hours, about 51 hours, about 52 hours).

In some aspects, provided herein are systems comprising a plurality of multilineage cardiovascular organoids, wherein each of the plurality of multilineage cardiovascular organoids comprises mesodermal and non-mesodermal cell types. In some embodiments, the plurality of multilineage cardiovascular organoids are generated by guided differentiation of a plurality of embryoid bodies derived from iPSCs of varying genotypes. In some embodiments, the systems described herein find use in a method of assessing cardiotoxicity of at least one agent or condition. In some embodiments, the method of assessing cardiotoxicity comprises contacting at least one of the plurality of multilineage cardiovascular organoids with the agent or subjecting at least one of the plurality of multilineage cardiovascular organoids to the condition and evaluating a response to the agent or the condition in at least one cell type within the at least one multilineage cardiovascular organoid. In some embodiments, evaluating a response to the agent or the condition in the at least one cell type within the at least one multilineage cardiovascular organoid comprises identifying one or more response expression quantitative loci (eQTLs) in the at least one cell type. In some embodiments, the one or more response eQTLs are identified using single-cell RNA sequencing (scRNA-seq).

In some aspects, provided herein are methods comprising exposing a multilineage cardiovascular organoid or a system comprising a plurality of multilineage cardiovascular organoids to an agent or condition, and evaluating a response to the agent or condition. In some embodiments, evaluating a response to the agent or condition comprises evaluating a response to the agent or the condition in at least one cell type within the multilineage cardiovascular organoid. In some embodiments, evaluating a response to the agent or condition comprises evaluating a response to the agent or condition in at least one cell type within at least one multilineage cardiovascular organoid in the system.

For any of the methods described herein, the agent can be a therapeutic agent for the treatment of a disease or condition. For example, the agent can be an anticancer agent. In some embodiments, the agent is an environmental pollutant, such as lead or mercury. In some embodiments, the multilineage cardiovascular organoid is subjected to a condition. In some embodiments, the condition is an atmospheric condition such as electromagnetic radiation, zero gravity, atmospheric pressure, and the like. In some embodiments, the condition is a physical condition, such as mechanical stress or agitation. In some embodiments, the condition is an environmental condition such a modulated temperature, humidity, oxygen content, CO2 content, etc.

### BRIEF DESCRIPTION OF THE DRAWING(S)

FIG. 1A-1C shows guided differentiation and characterization. FIG. 1A is a schematic showing an overview of the 10-5 guided differentiation protocol. CEPT: Chroman 1, emricasan, polyamines, and trans-ISRIB. HM: heart medium. Chiron: CHIR99021. FIG. 1B shows UMAP of 8,936 sequenced cells, manually annotated, alongside cell type proportions. FIG. 1C shows expression of canonical marker genes for each cell cluster.

FIG. 2 shows UMAP plots of cells from Seurat integration of a 16-day iPSC-CM differentiation time-course dataset and the guided differentiation dataset. Cells are colored according to cluster and dataset; grey cells are those present in the entire integrated dataset but not present in the selected dataset. The small plots represent cells from the differentiation time-course (n=230,849), plotted according to their day of collection. The large plot represents cells from a single collection of guided differentiation culture cells (n=8,936).

FIGS. 3A-3D show automated annotation using scPred trained on a fetal heart reference. FIG. 3A shows UMAP of fetal heart cells (n=13,569) with 11 cell types annotated by Miao et al. (2020)¹¹. FIG. 3B is a schematic showing an overview of automated annotation process using scPred. Model was trained using the cell reference and applied to three datasets, including peripheral blood mononuclear cells (PBMCs) used here as a non-cardiac control (FIG. 8) FIG. 3C shows a UMAP of classified cell types from the guided differentiation culture (n=5,391). FIG. 3D shows a UMAP of classified cell types from multi-lineage organoid published by Silva et al. (2021)¹³ (n=3,009).

FIG. 4A, FIG. 4B, and FIG. 4C show representative images of day 10 aggregates after guided differentiation of cell lines 19114, 19130, and 19152, respectively. Scale bar is 1000 microns.

FIG. 5A, FIG. 5B, and FIG. 5C show UMAP plots of individual cell lines, manually annotated, alongside overall proportion of each cell type. FIG. 5A shows cell line 19114, n=2,798. FIG. 5B shows cell line 19130, n=3,124. FIG. 5C shows cell line 19152, n=3,014.

FIG. 6A shows a UMAP plot of subclustered foregut endoderm cells (n=772). FIG. 6B shows violin plots comparing each cell subcluster for expression of posterior foregut marker genes. FIG. 6C shows violin plots comparing each cell subcluster for expression of anterior foregut marker genes.

FIG. 7 shows a heatmap of the top 10 genes (per cell cluster) according to differential expression (log fold change >2).

FIG. 8 shows a UMAP of classified cell types from peripheral blood mononuclear cells (PBMCs), used here as a non-cardiac control (n=15,297).

### DETAILED DESCRIPTION

Organoids comprise multiple cell types that self-organize and recapitulate major features of in vivo organs, including basic function, structural resemblance and constituent cell types. Such properties make organoids very useful as disease models. However, because organoid generation typically involves long-term (weeks or months) cell culture and differentiation procedures tailored to individual iPSC lines, organoids are usually not well-suited for population-level studies such as eQTL mapping efforts, which necessitate data collection from many (dozens or hundreds) individuals. Moreover, timecourse studies in traditional organoids are laborious and expensive. As such, improved methods for affordably and reliably generating organoids are needed.

In order to study cell-type-specific gene regulation at the population level, a “guided differentiation” approach was developed herein. This guided differentiation approach provides high cellular diversity, fast experimental throughput, and reproducibility across cell lines. Guided differentiation, as opposed to ‘directed’ differentiation, gently biases iPSCs towards the cardiac mesoderm lineage, while maintaining the greatest degree of cellular diversity possible. Within guided cultures, individual cells differentiate at dissimilar rates, permitting maximum diversity with respect to function (cell type) and time (cell differentiation stage). The inclusion of early progenitor cells, in addition to differentiated cells, provides continuous developmental trajectories for detecting transient genetic effects that typically can only be captured by using time-course experimental design. Herein it is demonstrated that guided cultures contain functionally diverse cell types, which represent multiple stages of cardiac differentiation, and that the transcriptional profiles of single cells in these cultures are comparable to that of single cells from mature cardiac organoids.

In some aspects, provided herein are multilineage cardiovascular organoids and methods of generating the same. The multilineage cardiovascular organoids described herein can be used for screening of agents, such as anti-cancer agents, for cardiotoxicity. For example, the multilineage cardiovascular organoids described herein can be used to quantify gene expression levels and identify response eQTLs that regulate transcriptional changes to agents (e.g. anti-cancer drugs) in multiple cell types, including multiple cardiovascular cell types. Response eQTLs (which are anchored by genotype) can provide a catalog of loci that interact either directly or indirectly with the treatment. Accordingly, identifying such response eQTLs reveals specific genes and pathways involved in normal cardiovascular function and indicates cardiotoxic mechanisms and associated genes that classify individual patients based upon susceptibility to cardiotoxicity.

Human induced pluripotent stem cells (iPSCs) can be differentiated from a variety of accessible tissues into cardiomyocytes, endothelial cells, and cardiac fibroblasts. However, differentiation protocols are often optimized for use in a single cell line, and their efficacy across a range of individuals is not guaranteed. Adequately powered molecular QTL mapping studies require dozens of individuals; thus, molecular QTL studies of differentiating cells are expensive, inefficient, and laborious to perform, which restricts the breadth of differentiated cell types and external treatments that can be realistically included in a single study. The impracticality of studying more than one cell type at a time is a major obstacle to understanding how individuals differ in their responses to anticancer drugs, which have widespread effects on multiple cardiovascular cell types. Moreover, organoids are laborious and expensive to generate, and in practice, inter-individual differences make it difficult to grow them consistently in dozens of individuals. This, organoids are typically impractical for population-level genomic analyses of cardiovascular cell types.

The multilineage cardiovascular organoids described herein allow for efficient growth and investigation of multiple cardiovascular cell types. The multilineage cardiovascular organoids described herein are advantageous over other methods previously used to model disease and test agents, as they provide the ability to grow multiple cardiovascular cell types in the same dish which obviates the need for complex differentiation protocols and allows for greater control over confounding variables that might mask genetic effects on gene expression.

### 1. Definitions

To facilitate an understanding of the present technology, a number of terms and phrases are defined below. Additional definitions are set forth throughout the detailed description.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “agent” and “drug” are used interchangeably herein in the broadest sense and refer to any substance that can be administered to a subject or is a potential candidate for administration to a subject, including as a therapeutic or a prophylactic. In some aspects, an “agent” refers to an anti-cancer agent or a candidate anti-cancer agent.

The term “cardiotoxicity” is used herein in the broadest sense and refers to any damage or dysfunction to the structure or function of the heart. For example, “cardiotoxicity” may refer to dysfunctional heart electrophysiology or damage to one or more tissues (e.g. muscles) of the heart. Generally, cardiotoxicity leads to a disruption of normal heart function and thereby causes inefficient circulation or blood in the body.

### 2. Multilineage Cardiovascular Organoids and Methods of Generating the Same

In some embodiments, provided herein are multilineage cardiovascular organoids. In some embodiments, provided herein are methods of generating multilineage cardiovascular organoids. A “multilineage cardiovascular organoid” may also be referred to herein as a “multilineage cardiovascular organoid tissue”, or a “multilineage cardiovascular organoid system”.

The term “organoid” as used herein refers to a structure that mimics one or more characteristics of an organ. A “multilineage cardiovascular organoid” refers to a three-dimensional structure that mimics one or more characteristics of the heart. For example, a multilineage cardiovascular organoid may comprise one or more cell types and/or tissue types present in the heart. For example, a multilineage cardiovascular organoid may comprise one or more cardiac cell types (e.g. cardiomyocytes, fibroblasts, vascular endothelial cells, mesodermal cells, non-mesodermal cells, etc.). In some embodiments, a multilineage cardiovascular organoid comprises multiple cardiac cell types. As another example, the structural organization of one or more cell/tissue types in a multilineage cardiovascular organoid may be similar to the organization seen in a heart in vivo. Finally, a multilineage cardiovascular organoid may also mimic one or more functions of a heart, such as exhibiting a spontaneous response to various drugs/compounds.

In some embodiments, provided herein is a multilineage cardiovascular organoid comprising a plurality of cardiac cell types and/or cardiac tissue types. In some embodiments, the multilineage cardiovascular organoid comprises mesodermal cell types. In some embodiments, the multilineage cardiovascular organoid comprises beating cardiomyocytes, fibroblasts, and vascular endothelial cells. In some embodiments, the multilineage cardiovascular organoid additionally comprises other mesodermal cell types. In some embodiments, the multilineage cardiovascular organoid additionally comprises non-mesodermal cell types, including gut endoderm and cardiac neural crest cells. These tissue types are valuable for cardiac modeling; for instance, may support cardiac cell differentiation and neural crest cells give rise to cardiac tissues.

In some embodiments, the multilineage cardiovascular organoids described herein comprise one or more of the following cell types: cardiac progenitor cells, proliferating cells, cardiomyocytes, fibroblasts, myofibroblasts, endoderm epithelium, foregut, epicardium, endocardium, ectoderm, and neural crest cells.

In some embodiments, the multilineage cardiovascular organoids described herein comprise cardiac progenitor cells. Cardiac progenitor cells can be identified based upon one or more suitable markers, including but not limited to ISL1, NKX2-5, and PDGFRA.

In some embodiments, the multilineage cardiovascular organoids described herein comprise proliferating cells. Proliferating cells can be identified based upon one or more suitable markers, including but not limited to cell cycle genes MKI67 and UBE2C.

In some embodiments, the multilineage cardiovascular organoids described herein comprise cardiomyocytes. Cardiomyocytes can be identified based upon one or more suitable markers, including but not limited to sarcomere genes (TNNT2 and ACTN2) and ion channel genes (SLC8A1 and KCNH7). In some embodiments, the multilineage cardiovascular organoids described herein comprise mature cardiomyocytes. Mature cardiomyocytes can be identified based upon expression of suitable markers, such as TNNI3 and MYH7. In some embodiments, the multilineage cardiovascular organoids described herein comprise cardiomyocyte subtypes, such as ventricular, atrial, and nodal cardiomyocytes. Ventricular cardiomyocytes can be identified by expression of markers such as FHL2 and IRX4, atrial cardiomyocytes can be identified based upon expression of markers such as MYL7 and NPPA. Nodal cardiomyocytes can be identified based upon expression of markers, including HCN1 and HCN4. Smooth muscle cells may be identified based on expression of cell markers, such as ACTA2, TAGLN, and CNN1.

In some embodiments, the multilineage cardiovascular organoids described herein comprise fibroblasts and/or myofibroblasts. Fibroblasts and myofibroblasts can be identified, for example, by using expression of COL3A1 as well as the canonical fibroblast markers PDGFRA and TCF21. Myofibroblasts can be distinguished from fibroblasts by expression of suitable markers such as ACTA2, DCN, and LUM. Additionally, myofibroblasts can be identified based upon reduced expression of PDGFRA and TCF21 but increased expression of COL3A1 relative to fibroblasts.

In some embodiments, the multilineage cardiovascular organoids described herein comprise endoderm lineage cells. Endoderm lineage cells may comprise endoderm epithelial cells, which may be identified for example based upon expression of EPCAM and CDH1. Endoderm lineage cells may also comprise foregut cells, including liver cells and intestinal cells. Liver cells may be identified by expression of AFP and SERPINA1, for example, Intestinal cells may be identified by expression of suitable markers, including APOB. Endoderm may support cardiomyocyte differentiation.

In some embodiments, the multilineage cardiovascular organoids described herein comprise and ectoderm lineage clusters. Ectoderm cells can be identified using, for example, PAX6 expression.

The multilineage cardiovascular organoids described herein may also comprise neural crest cells. Neural crest cells can be identified, for example, by using PAX3 expression.

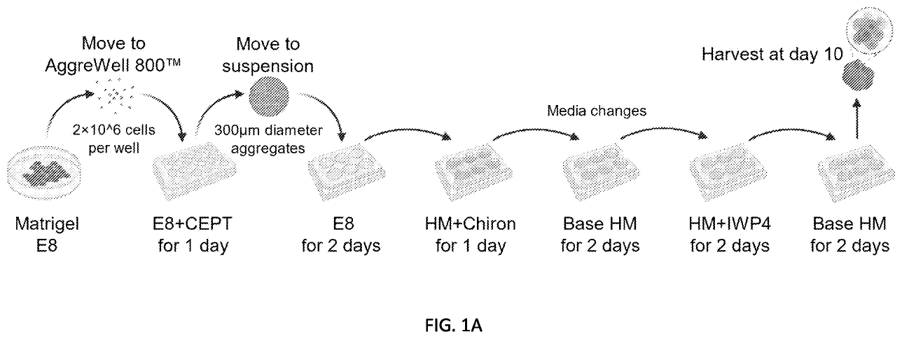

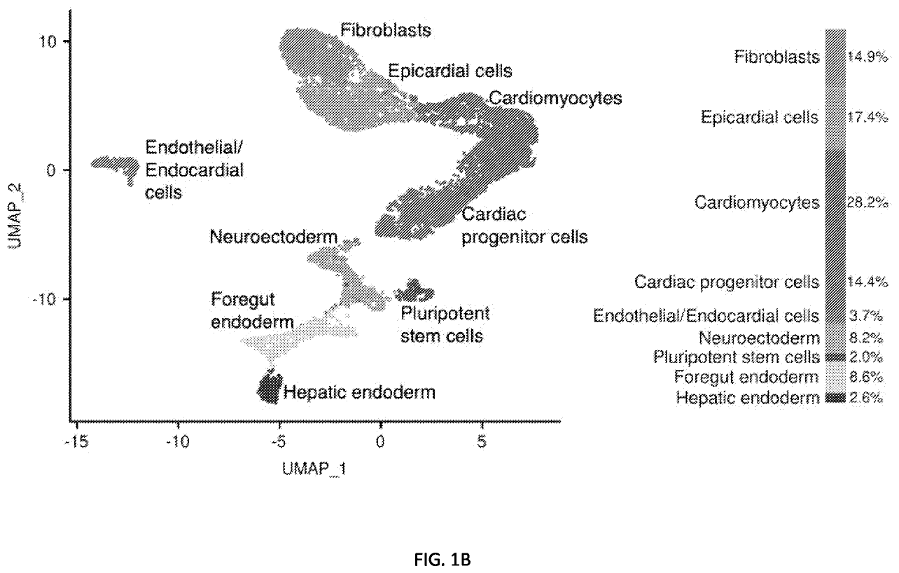

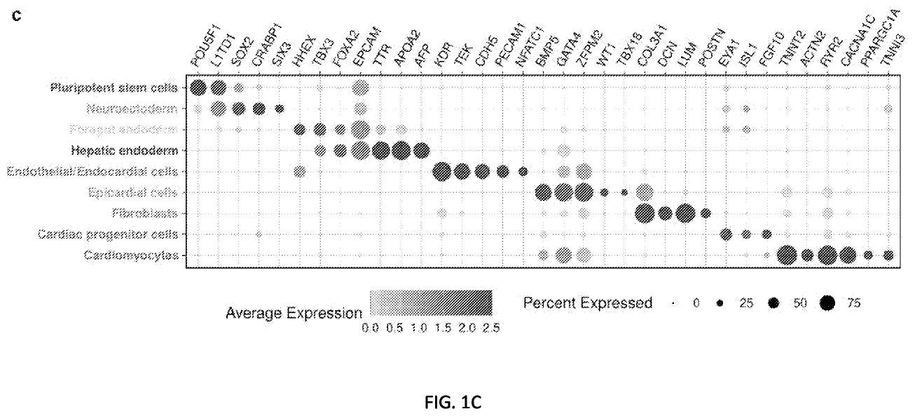

Generally, the cell type composition depends on the day that the cardiac organoids are harvested. In some embodiments, performing guided differentiation through day 10 (e.g. day 10 overall, calculated by the method shown in FIG. 1) yields 9 different cell types: fibroblasts, epicardial cells, cardiomyocytes, cardiac progenitor cells, endothelial/endocardial cells, neuroectoderm, pluripotent stem cells, foregut endoderm, and hepatic endoderm. In some embodiments, performing guided differentiation for an additional 2 days, i.e., harvesting at day 12, yields 10 different cell types: epicardial cells, cardiomyocytes, cardiac neural crest cells, cardiac progenitor cells, endothelial/endocardial cells, neuroectoderm, foregut endoderm, hepatic endoderm, and 2 distinct populations of fibroblast cells. Note that, relative to day 10 cardiac organoids, the day 12 cardiac organoids lack the population of pluripotent stem cells but gain 2 new cell populations: cardiac neural crest and a second fibroblast population.

In some embodiments, provided herein are systems comprising a plurality of multilineage cardiovascular organoids. In some embodiments, each of the plurality of organoids comprises mesodermal and non-mesodermal cell types, including those described above.

In some embodiments, provided herein is a method for generating one or more multilineage cardiovascular organoids. In some embodiments, the method comprises culturing an embryoid body in a cardiac induction medium comprising a Wnt signaling activator. In some embodiments, the method further comprises culturing the embryoid body in a Wnt signaling inhibitor medium comprising a Wnt signaling inhibitor. In some embodiments, the method comprises culturing an embryoid body in a cardiac induction medium comprising a Wnt signaling activator followed by culturing the embryoid body in a Wnt signaling inhibitor medium comprising a Wnt signaling inhibitor.

In some embodiments, the method for generating one or more multilineage cardiovascular organoids comprises first generating the embryoid body. The term “embryoid body” (or “EB”) refers to a three-dimensional organoid that spontaneously and asynchronously differentiates into all three germ layers (e.g. endoderm. mesoderm, and ectoderm). Suitable methods for generating embryoid bodies and uses of embryoid bodies for in vivo studies are disclosed in Rhodes et al., (2022) Human embryoid bodies as a novel system for genomic studies of functionally diverse cell types, eLife 11: e71361, the entire contents of which are incorporated herein by reference for all purposes. The methods described therein are intended to be one example of how embryoid bodies may be generated, other suitable methods for generating embryoid bodies may alternatively be used.

The embryoid body may be derived from stem cells. For example, the embryoid body may be derived from pluripotent stem cells. The term “pluripotent stem cells” refers to stem cells are cells that have the capacity to self-renew by dividing, and to develop into the three primary germ cell layers of the early embryo and therefore into all cells of the adult body. Examples of pluripotent stem cells include embryonic stem cells and induced pluripotent stem cells. In some embodiments, the embryoid body is derived from (e.g. differentiated from) induced pluripotent stem cells (iPSCs). For example, in some embodiments the method comprises differentiating pluripotent stem cells (e.g. iPSCs).

The pluripotent stem cells (e.g. iPSCs) maybe from any suitable species. In some embodiments, the pluripotent stem cells are human cells. In some embodiments, the pluripotent stem cells are from a mammal other than a human. Nonlimiting examples of suitable mammals from which pluripotent stem cells may be derived include non-human primates, cats, dogs, pigs, horses, sheep, rodents (e.g. mice, rats, gerbils, hamsters), and the like.

The pluripotent stem cells (e.g. iPSCs) may be of any suitable genotype. One advantage of the present disclosure is that a system is provided comprising a plurality of multilineage cardiovascular organoids, wherein the multilineage cardiovascular organoids are generated by guided differentiation of a plurality of embryoid bodies derived from iPSCs of varying genotypes. Accordingly, the system can be used to model response to an agent (e.g. an anti-cancer agent) in organoids derived from subjects having genetic variability. This allows for personalized medicine, such as the selection of agents (e.g. anti-cancer agents) for a subject based upon a genetic assessment of cardiotoxicity risk.

In some embodiments, the pluripotent stem cells (e.g. iPSCs) are derived from a subject (including human and non-human subjects, as described above) suffering from a disease or condition. In some embodiments, the use of pluripotent stem cells derived from a subject suffering from a disease or condition generates a multilineage cardiovascular organoid model of the disease or condition. In some embodiments, the pluripotent stem cells derived from a subject suffering from the disease or condition generates a multilineage cardiovascular organoid wherein disease-related genetic variation is present. In some embodiments, the pluripotent stem cells are derived from healthy subjects. In some embodiments, the response of a multilineage cardiovascular organoid generated from pluripotent stem cells from a subject having a disease or condition (e.g. heart disease, cancer, etc.) to an agent is compared to the response of a multilineage cardiovascular organoid generated from pluripotent stem cells from a healthy subject to identify disease-associated genetic variations.

In some embodiments, the pluripotent stem cells are derived from a subject suffering from a heart disease, and therefore can be used to generate a multilineage cardiovascular organoid model of the heart disease. In some embodiments, the subject does not have a heart disease. In some embodiments, the subject has cancer. For example, in some embodiments, the iPSCs are derived from a subject suffering from cancer, and therefore can be used to predict cardiotoxicity of an anti-cancer agent in the subject prior to administration of the agent.

In some embodiments, the method for generating a multilineage cardiovascular organoid comprises biasing EBs to differentiate along the cardiac lineage. In some embodiments, the method comprises differentiating iPSCs to generate one or more embryoid bodies, and guiding the one or more embryoid bodies towards the cardiac lineage. Biasing iPSCs to generate EBs biased towards cardiac lineage is also referred to herein as a “guided differentiation” or a “guided differentiation approach”. Accordingly, in some embodiments the method for generating a multilineage cardiovascular organoid comprises guiding differentiation of an embryoid body to a cardiac lineage. This is in contrast to a “directed differentiation” or a “directed differentiation approach”. The guided differentiation approach described herein produces a population of disorganized cell aggregates comprised of asynchronously differentiating cells from different stages of cardiac development, including beating cardiomyocytes, cardiac progenitors, fibroblasts, and endothelial cells. The methods described herein reproducibly and efficiently generate diverse cardiovascular cell populations from different individuals. Moreover, the ability to grow multiple cardiovascular cell types in the same dish obviates the need for complex differentiation protocols and allows for greater control over confounding variables that might mask genetic effects on gene expression—which is useful for detecting gene-by-drug interactions. It also provides an opportunity to study cell type-specific responses to anticancer drugs. The use of these multilineage cardiovascular organoids therefore circumvents many of the challenges associated with differentiated cells and allows for mapping drug response ecQTLs in multiple cell types from dozens of individuals.

In some embodiments, iPSCs are differentiated into embryoid bodies and biased towards a cardiac lineage by using a guided differentiation approach. In a matter of days, this produces a population of differentiating cells from different stages of cardiovascular differentiation, including beating cardiomyocytes, vascular endothelial cells, and cells from non-mesodermal tissues (e.g., foregut) that support cardiac modeling. For example, these cell types are shown in FIG. 1B and FIG. 1C.

In some embodiments, the method for generating a cardiac embryoid described herein comprises aggregating stem cells (e.g. iPSCs) into embryoid bodies (EBs). In some embodiments, stem cells are aggregated into EBs on a suitable surface. In some embodiments, the surface comprises a cell culture plate. In some embodiments, the cell culture plate comprises one or more microwells. In some embodiments, the one or more microwells are about 200-1000 microns in diameter. For example, in some embodiments the one or more microwells are about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, about 1000 microns. An exemplary cell culture plate is an AggreWell™ 800 Microwell culture plate, commercially available from STEMCELL™ Technologies. In some embodiments, stem cells are aggregated into EBs on a suitable surface using a suitable medium to generate embryoid bodies having a diameter of about 200 to 400 microns. In some embodiments, the embryoid bodies have a diameter of about 300 microns.

In some embodiments, the method for generating a multilineage cardiovascular organoid described herein comprises generating an embryoid body from induced pluripotent stem cells, and guiding differentiation of the embryoid body to a cardiac lineage. In some embodiments, the generated EBs (e.g. the stem cells aggregated into EBs on a suitable cell culture plate, as described above) are biased towards (e.g. guided towards) a cardiac lineage using a suitable culture medium. In some embodiments, guiding EBs towards a cardiac lineage comprises culturing the EBs in a first stem cell culture medium. A stem cell culture medium may be any medium that supports the growth and health of stem cells. In some embodiments, the stem cell culture medium is a feeder-free medium, such as Essential 8™ or MTeSR™ medium, although other suitable cell culture mediums may be used. In particular, culture mediums suitable for iPSCs are preferable.

In some embodiments, the EBs are cultured in the first stem cell culture medium for a suitable first duration of time on a first surface, and are then transferred to a second surface and cultured for a second duration of time. Being cultured “on” or transferred “to” a surface does not necessarily indicate that the cells adhere or are adhered to said surface. For example, cells may be transferred from a formalin plate to an ultra-low attachment plate, and although the cells do not adhere to the ultra-low attachment plate they are still considered to have been transferred “to” the ultra-low attachment plate. In some embodiments, EBs are cultured in the first stem cell culture medium (e.g. E8 medium) for a first duration of time on a formation plate, and are then transferred to an ultra-low attachment plate. In some embodiments, the second surface (e.g. ultra-low attachment plate) comprises a first cell culture medium (e.g. E8 medium). For example, in some embodiments the generated EBs are cultured in E8 medium for a first duration of time on a formation plate before transferring them to ultra-low attachment plates containing E8 medium. In some embodiments, the EBs are cultured for 20-28 hours on the first surface (e.g. the formalin plate) and for about 40-60 hours on the second surface (e.g. on the ultra-low attachment plate). In some embodiments, the EBs are cultured on the first surface for about 24 hours and are cultured on the second surface for about 48 hours.

In some embodiments, biasing EBs towards the cardiac lineage comprises culturing the EBs in a cardiac induction medium. In some embodiments, following culture for a suitable duration of time with the first stem cell culture medium, the medium is exchanged for a cardiac induction medium. The term “cardiac induction medium” as used herein refers to a cell culture medium containing the basic nutrients necessary to promote cell health and growth (e.g. nutrients, amino acids, vitamins, etc., also referred to herein as a “basal medium” or “basal heart medium”) along with suitable reagents to induce differentiation of EBs to the cardiac lineage. In some embodiments, the cardiac induction medium comprises basal heart medium and a Wnt signaling activator. In some embodiments, the Wnt signaling activator is a small molecule Wnt signaling activator. Exemplary small molecule Wnt signaling activators include, but are not limited to, SFRP inhibitors (e.g. WAY-316606), Notum inhibitors (e.g. ABC99), PP2A activators (e.g. IQ1), ARFGAP1 activators (e.g. QS11), GSK3 inhibitors (e.g. SB-216763, CHIR99021, BIO(6-bromoindirubin-3′-oxime), LY2090314), and beta-catenin activators (e.g. DCA). In some embodiments, the small molecule Wnt signaling activator is a GSK3 inhibitor. In some embodiments, the Wnt signaling activator is CHIR99021.

In some embodiments, the cardiac induction medium comprises the basic nutrients necessary to promote cell growth and health (e.g. a basal medium as described above, such as RPMI 1640 medium) and additionally comprises the Wnt signaling activator (e.g. CHIR99021). In some embodiments, the cardiac induction medium comprises the Wnt signaling activator (e.g. CHIR99021) at a concentration of about 1-10 μM. In some embodiments, the cardiac induction medium comprises the Wnt signaling activator at a concentration of about 1 μM, 2 μM, 3 μM, 1 μM, 4 μM, 5 μM, 6 μM, 7 μ, 8 μM, 9 μ, or about 10 μM. In some embodiments, the cardiac induction medium comprises CHIR99021 at a concentration of about 4 μM. In some embodiments, in addition to the Wnt signaling activator, the cardiac induction medium comprises one or more supplements (e.g. B-27 supplement minus insulin), antibiotics, and the like. For example, in some embodiments the cardiac induction medium comprises RPMI 1640, B-27 minus insulin, and a Wnt signaling activator (e.g. CHIR99021).

In some embodiments, the EBs are cultured for a suitable duration of time in the cardiac induction medium. In some embodiments, the EBs are cultured for about 12 hours to about 48 hours in the cardiac induction medium, which initiates differentiation along the cardiac lineage. In some embodiments, the EBs are cultured for about 24 hours in the cardiac induction medium.

In some embodiments, the method further comprises culturing the EBs in a basal heart medium. The term “basal heart medium” or “basal medium” refers to a cell culture medium containing the basic nutrients necessary to promote cell health and growth (e.g. nutrients, amino acids, vitamins, etc.). In some embodiments, following culture with the cardiac induction medium the EBs are cultured in a basal heart medium. The basal heart medium may comprise the same components present in the cardiac induction medium, but lack the Wnt signaling activator. For example, the basal heart medium may comprise RPMI 1640, and B-27, and not comprise the Wnt signaling activator. In some embodiments, the basal heart medium comprises B-27 without insulin. In some embodiments, the EBs are cultured in the basal heart medium for 48-72 hours. In some embodiments, the EBs are cultured in the basal heart medium for about 48 hours. For example, in some embodiments after a suitable duration of time (e.g. after 24 hours), the cardiac induction medium is replaced with a basal heart medium lacking the Wnt signaling activator.

In some embodiments, the method further comprises culturing EBs in a Wnt signaling inhibitor medium. In some embodiments, the Wnt signaling inhibitor medium comprises the basal heart medium and additionally comprises a Wnt signaling inhibitor. In some embodiments, the Wnt signaling inhibitor is a porcupine inhibitor (e.g. LGK974, C59, ETC-159, IWP-4, IWP-2), a Frizzled inhibitor (e.g. niclosamide, peptide), an axin activator (IWR, G007-LK, G244-LM, XAV939), or a TCF inhibitor.

In some embodiments, the Wnt signaling inhibitor is a porcupine inhibitor. In some embodiments, the Wnt signaling inhibitor is IWP-4. In some embodiments, the Wnt signaling inhibitor medium comprises RPI 1640, B-27 minus insulin, and IWP-4. In some embodiments, the Wnt signaling inhibitor medium comprises the Wnt signaling inhibitor (e.g. IWP-4) at a concentration of about 0.5 μM, 1 μM, 1.5 μM, 2 μM, 2.5 μM, 3 μM, 3.5 μM, 4 μM, 4.5 μM, μM, or 5 μM. In some embodiments, the Wnt signaling inhibitor medium comprises the Wnt signaling inhibitor (e.g. IWP-4) at a concentration of about 2 μM.

In some embodiments, the method further comprises culturing the EBs in a basal heart medium following culture in the Wnt signaling inhibitor medium. Accordingly, in some embodiments the EBs are cultured in a basal heart medium at least two times, once after culture in the cardiac induction medium and once again after culture in the Wnt signaling inhibitor medium. In some embodiments the EBs are cultured in a basal heart medium following culture in the Wnt signaling inhibitor medium for 2 days, at which point the differentiated EBs (which are now formed multilineage cardiovascular organoids) are harvested. In some embodiments, the EBs are cultured in a basal heart medium following culture in the Wnt signaling inhibitor medium for about 5-7 days, at which point the differentiated EBs are harvested. In some embodiments, the basal heart medium is refreshed every 24 hours, every 48 hours, or every 72 hours until the differentiated EBs arc harvested.

An exemplary timeline for a method for generating a multilineage cardiovascular organoid is shown in FIG. 1A. In some embodiments, a method for generating a multilineage cardiovascular organoid comprises forming embryoid bodies, and then guiding differentiation of the embryoid bodies to form multilineage cardiovascular organoids. In some embodiments, embryoid bodies are formed by culturing stem cells, such as iPSCs, in a suitable cell culture medium and then aggregating the stem cells on a suitable plate. For example, in some embodiments embryoid bodies are formed by culturing iPSCs in a suitable medium until a desired confluency has been achieved. For example, as shown in FIG. 1A in some embodiments iPSCs are cultured in Matrigel Growth Factor Reduced (GFR) Basement Membrane Matrix with Essential 8™ (E8) medium and suitable antibiotics (e.g. Penicillin-Streptomycin). In some embodiments, at roughly 80% confluency iPSCs are passaged using a dissociation reagent and seeded onto an aggregation plate. For example, in some embodiments the cells are disassociated and seeded onto a multi-well plate (e.g. AggreWell™800 plate) coated with an anti-adherence agent. In some embodiments, iPSCs are disassociated and seeded onto the aggregation plate using E8 medium with one or more cytoprotectants. Suitable cytoprotectants include, for example, a Rho-kinase inhibitor (E.g. Y-27632) and CEPT. In some embodiments, iPSCs are dissociated and seeded onto the aggregation plate using E8 medium with CEPT (i.e., chroman 1, emricasan, polyamines, and trans-ISRIB). According to the timeline shown in FIG. 1A, the day of dissociation and seeding onto the aggregation plate is considered “day 0” for the method of generating a multilineage cardiovascular organoid. In some embodiments, the method comprises allowing cell aggregates to form on the aggregation plate. In some embodiments, cell aggregates having a diameter of about 300 μm form. In some embodiments, about 2 million cells per well are plated on the aggregation plate, thus allowing formation of aggregates having a diameter of about 300 μm. In some embodiments, the plate is centrifuged to aggregate cells together. For example, in some embodiments the plate is centrifuged at about 100-200 g for about 2-5 minutes. In some embodiments, embryoid bodies are kept on the plate for a suitable duration of time, such as 24 hours, and are then transferred to a suspension culture in a suitable medium, such as E8 medium. In some embodiments, the suspension culture is an ultra-low adherence plate. In some embodiments, cells are kept in the suspension culture for 48 hours. The term “embryoid bodies” is inclusive of the aggregated cells on the aggregation plate and the aggregated cells in suspension culture.

In some embodiments, after culturing the embryoid bodies in the suspension culture for 48 hours, guided differentiation of the embryoid bodies is performed. As shown in FIG. 1A, in some embodiments guided differentiation comprises changing the suspension culture medium (e.g. E8 medium) to a cardiac induction medium. In some embodiments, the suspension culture medium is changed to cardiac induction medium on day 3. In some embodiments, the cardiac induction medium comprises basal heart medium plus 4 μM CHIR99021. In some embodiments, the basal heart medium comprises RPMI 1640 Medium, GlutaMAX™ Supplement, HEPES (Thermo Fisher Scientific, 72400047), 2% v/v B-27 Supplement, minus insulin (Thermo Fisher Scientific, A1895601), and Penicillin-Streptomycin. In some embodiments, after 24 h (e.g. on day 4) the cardiac induction medium (heart medium+CHIR99021) is changed to basal heart medium for 48 h. In some embodiments, on day 6 basal medium is changed to heart medium plus 2 μM IWP-4 for 48 h. In some embodiments, on day 8, medium is changed to basal heart medium for 48 h followed by collection of multilineage cardiovascular organoids on day 10. In some embodiments, contraction of the multilineage cardiovascular organoid becomes visible on day 8, after incubation with cardiac induction medium (e.g. Wnt activation) and after Wnt inhibition (e.g. incubation with IWP-4). The term “multilineage cardiovascular organoid” includes the organoids present from at the time point when contraction becomes visible onwards. For example, in some embodiments the term “multilineage cardiovascular organoid” includes the organoids present on day 8, day 9, or day 10 relative to FIG. 1A. However, visible contraction is not required to meet the definition of a multilineage cardiovascular organoid. In some embodiments, a “multilineage cardiovascular organoid” refers to a group of cells that have been cultured in cardiac induction medium (e.g. basal heart medium plus Wnt signaling activator) followed by basal heart medium. In some embodiments, a multilineage cardiovascular organoid refers to a group of cells that have been cultured in cardiac induction medium, followed culture in by basal heart medium, followed by culture in basal heart medium with a Wnt signaling inhibitor. In some embodiments, a multilineage cardiovascular organoid refers to a group of cells that have been cultured in cardiac induction medium, followed culture in by basal heart medium, followed by culture in basal heart medium with a Wnt signaling inhibitor, followed by culture in a basal heart medium.

In some embodiments, the culture temperature may be about 30 to 40° C., and preferably about 37° C, although other suitable temperatures may be used. In some embodiments, the concentration of CO₂ is about 1 to 10%, such as about 2 to 5%. In some embodiments, the oxygen concentration is about 1% to 30%. In some embodiments, the oxygen concentration is about 5% to about 25%. In some embodiments, the oxygen concentration is about 10% to about 25%. In some embodiments, the oxygen concentration is from about 15% to about 25%. For example, in some embodiments the oxygen concentration is about 1%, about 5%, about 10%, about 15%, about 20%, about 25%, or about 30%. In some embodiments, the oxygen concentration is about 21%.

In some embodiments, provided herein is a system comprising a plurality of multilineage cardiovascular organoids as described herein. In some embodiments, the plurality of multilineage cardiovascular organoids are generated by guided differentiation of embryoid bodies. In some embodiments, the plurality of multilineage cardiovascular organoids are generated by guided differentiation of a plurality of embryoid bodies derived from iPSCs of varying genotypes. Exemplary methods for generation of embryoid bodies, and for guided differentiation of embryoid bodies, are described above.

The multilineage cardiovascular organoids and systems described herein find use in a variety of methods. These methods involve exposing the multilineage cardiovascular organoids or systems described herein to an agent or condition, and evaluating a response to the agent or condition. In some embodiments, provided herein is a method comprising exposing a multilineage cardiovascular organoids or at least one multilineage cardiovascular organoid in a system described herein to an agent or condition, and evaluating a response to the agent or condition in at least one cell type within a multilineage cardiovascular organoid. In some embodiments, the method is conducted to evaluate toxicity of an agent. In some embodiments, the method is conducted to evaluate cardiotoxicity of a potential therapeutic agent, such as a potential anti-cancer agent. In some embodiments, the method is conducted to evaluate efficacy of an agent, such as a potential therapeutic agent.

For example, drug response testing (for both toxicity and efficacy) and environmental response testing can be performed using the multilineage cardiovascular organoids described herein. For example, multilineage cardiovascular organoids can be treated with an agent (e.g. a chemical, a compound) and/or an environmental exposure, and a generalized and/or cell type specific response to treatment can be evaluated. For example, efficacy can be assessed by analyzing change in expression of target genes and pathways in target cell types. As another example, toxicity can be evaluated by identifying cell type specific patterns of cell death and cell type specific upregulation of genes associated toxicity, including genes associated with stress and apoptosis. Moreover, patterns of gene expression associated with cell type specific drug toxicity can be evaluated by collecting single-cell RNA-seq data from multilineage cardiovascular organoids treated with drugs with known toxicities in vivo in humans and extracting insights using machine learning algorithms. Similarly, multilineage cardiovascular organoids can be treated with combinations of combinations of drugs and environmental exposures. This can reveal drug interactions and drug-environment interactions. Associations between these interactions with efficacy and toxicity can be identified, along with genetic variants associated with response to these interactions.

In some embodiments, the multilineage cardiovascular organoids described herein are used to assess drug efficacy, assess toxicity of a drug or condition, and/or identify novel targets (e.g. target genes) for therapeutic intervention (e.g. novel drug targets). Drug response in organoids can be characterized in organoids by changes in cellular morphology, growth and viability, along with differences in levels of RNA transcripts, and specific proteins or metabolites, after contacting the multilineage cardiovascular organoid with the drug. Response magnitude can be tissue-specific or even cell type-specific. Further, inter-individual variation in drug response can be partially attributed to differences in genetic background. Therefore, drug response characterization should be performed using relevant cell types across a genetically diverse group of individuals. The multilineage cardiovascular organoids described herein provide an excellent platform for assessing drug response. For example, for drugs where a mechanism of action is known or gene response is known, the multilineage cardiovascular organoids described herein can be used to assess whether the multilineage cardiovascular organoid responds to a given agent in a manner consistent with what is expected (e.g. consistent with the known mechanism of action, consistent with the known gene response, etc.). Accordingly, individuals sensitive or resistant to a treatment can be identified based upon whether the response of a multilineage cardiovascular organoid generated from an individual is as expected or differs from what is expected. For example, a multilineage cardiovascular organoid generated from an individual of a given genotype may differ from what is an expected response to a given agent or condition, and as such that individual may be identified as either sensitive or resistant to the treatment based upon that response.

In some embodiments, the multilineage cardiovascular organoids described herein in methods of drug target discovery. For example, using single-cell RNA-sequencing, gene expression across cell types can be compared between multilineage cardiovascular organoids from healthy individuals and diseased individuals. Genes differentially expressed between these two groups represent potential drug targets. Moreover, gene expression differences between these two groups, across cell types, can help to determine the causal cell type and reveal unknown disease mechanisms.

By identifying new context-specific eQTLs, dynamic eQTLs, and response eQTLs, previously unknown gene regulatory landscapes can be identifying. By identifying novel mechanisms of regulation for disease-relevant genes and proteins, novel mechanisms through which to modulate expression of these targets can be found.

In some embodiments, the multilineage cardiovascular organoids and systems described herein are used to assess cardiotoxicity of at least one agent or at least one condition. In some embodiments, assessing cardiotoxicity of the agent or condition comprises contacting the multilineage cardiovascular organoid or system to the agent or condition, and evaluating the response of one or more cell types within the multilineage cardiovascular organoid to the agent or condition. In some embodiments, different cell types exhibit different reactions, including different reactions at differing time points, following contact with the agent or following exposure to the condition. Accordingly, the multilineage cardiovascular organoids described herein allow for cell type-specific responses to external perturbations, and measurement of how these responses change over time and/or between individuals with different genotypes.

In some embodiments, the multilineage cardiovascular organoids and systems described herein are used in a method of assessing cardiotoxicity of an agent. The cardiotoxicity of any suitable agent may be evaluated using the multilineage cardiovascular organoids and systems described herein. Suitable agents include, for example, therapeutic agents for any number of disorders or conditions. The disclosure is not intended to be limited to any particular agent, or any particular disorder or condition. In some embodiments, the agent is an anticancer agent. Anticancer drug-induced cardiovascular toxicity (CT) is a major side effect for many patients undergoing treatment for oncological disorders. CT symptoms vary widely across individuals, both in presentation and in time to onset. Risk of CT complicates treatment protocols and places cancer patients under additional duress. Moreover, little is known about the genetic factors driving inter-individual differences in responses to anticancer drugs. To understand why certain individuals are susceptible to drug-induced CT, an understanding of how anticancer drugs behave in cardiovascular cell types from different genetic backgrounds is needed. Specifically, to understand the genetic basis for drug-induced CT, it is significant to understand how anticancer drugs stimulate transcriptomic responses in multiple cardiovascular cell types from individuals of different genetic backgrounds. This can be accomplished using the multilineage cardiovascular organoids described herein.

Although an anticancer agent is one exemplary agent that can be assessed using the multilineage cardiovascular organoids described herein, the methods described herein are not to be construed as limited to evaluating anticancer agents. Any suitable agent may be used, including therapeutic agents for a variety of diseases or conditions. In some embodiments, the multilineage cardiovascular organoids and systems described herein are used to assess the cardiotoxicity of a condition, such as an environmental condition, a physical condition, etc. The multilineage cardiovascular organoids can be subjected to any conceivable condition in order to evaluate the effect of said condition on the organoids. For example, the multilineage cardiovascular organoids described herein can also be subjected to environmental pollutants (e.g., lead or mercury). As another example, the multilineage cardiovascular organoids can be subjected to physical conditions such as mechanical stress, atmospheric conditions such as low oxygen, or other environmental conditions such as electromagnetic radiation, zero gravity, and the like.

In some embodiments, the methods provided herein involve contacting a multilineage cardiovascular organoid with an agent or subjecting the multilineage cardiovascular organoid to a condition, and evaluating a response to the agent or the condition in at least one cell type within the multilineage cardiovascular organoid. In some embodiments, evaluating a response to the agent or condition comprises measuring gene expression, activity, or regulation in the at least one cell type. A variety of suitable methods may be performed to evaluate the response to the agent or condition in the at least one cell type within the multilineage cardiovascular organoid. For example, in some embodiments the response to the agent or the condition in the at least one cell type within the multilineage cardiovascular organoid is evaluated by measuring gene expression by PCR-based techniques or sequencing, evaluating chromatin accessibility, measuring one or more epigenetic markers, or evaluating expression quantitative loci (eQTLs).

In some embodiments, the multilineage cardiovascular organoids described herein can be used to identify loci that regulate gene expression levels in the cardiovascular system in response to an agent. Loci that regulate gene expression levels are referred to as “expression quantitative trait loci”, or “eQTLs”. Loci that regulate gene expression levels (eQTLs) (e.g. in the cardiovascular system) in response to an agent are referred to as response eQTLs. Response eQTLs are likely to underlie functional differences between individuals in response to different conditions or contexts. Generally, eQTLs—because they have functional consequences-are easier to interpret than random loci associated with clinical phenotypes, and in some cases, they can help to explain disease risk. For example, hundreds of loci have been identified that regulate the transcriptional response of differentiated cardiomyocytes to the anthracycline doxorubicin (DOX), an anticancer drug with known cardiotoxic effect. Notably, different cell types can vary in their response eQTLs to anticancer drugs. In some embodiments, the multilineage cardiovascular organoids provided herein can be used to map loci that regulate transcript levels in response to drug treatment. Accordingly, the multilineage cardiovascular organoids provided herein can be used to identify genetic factors that modulate cardiotoxicity (CT) risk. This allows clinicians to provide personalized treatment based on patient genotype. Accordingly, in some embodiments provided herein is a high-throughput model system comprised of multiple cardiac cell types that can be used to identification of CT-associated response eQTLs.

In some embodiments, RNA sequencing is used to evaluate gene expression in single cell types within the organoid. This is referred to herein as “single-cell RNA-seq” or “scRNA-seq”. Single-cell RNA-seq (scRNA-seq) can be used to deconvolve multilineage cardiovascular organoids into their component cell types, identify cell type-specific responses to external perturbations, and measure how these responses change between individuals with different genotypes.

In some embodiments, scRNA-seq can be used to identify response eQTLs. In some embodiments, scRNA-seq is used to measure the responses of multiple cardiovascular cell types to different classes of drugs, such as different classes of anticancer drugs. In some embodiments, scRNA-seq is used to measure the response of multiple cardiovascular cell types and other cell types within the multilineage cardiovascular organoid to different classes of drugs, such as different classes of anticancer drugs.

In some embodiments, response eQTLs, or genetic variants associated with the likelihood of transcriptional drug response and/or toxicity, can be identified using single-cell RNA sequencing. Response eQTLs can be called using a variety of differing techniques. For example, eQTLs can be called using pseudobulk data from cells assigned to a “cell type” via clustering and marker gene analysis. Alternatively, eQTLs can be called using gene expression measurements from individual cells. In cases where a single genetic variant has a large effect on drug toxicity, presence or absence of that variant can be used to stratify the ideal patient population and to inform decisions about which patients receive the drug or what dose will be most appropriate.

Often, many genetic variants will have a small effect of drug response. Using the effects sizes of response eQTL analysis, a Polygenic Risk Score can be constructed. For example, for a given patient, a polygenic risk score can be calculated, representing their individual likelihood for drug toxicity or efficacy based on their genetic background. PRS can then be used to inform treatment decisions or to recruit the ideal patient population for clinical trials.

The following examples further illustrate the invention but, of course, should not be construed as in any way limiting its scope.

### EXAMPLES

### Example 1

In some embodiments, provided herein is a novel, high-throughput multilineage cardiovascular organoid system. The system was developed using a panel of stem-cell-derived embryoid bodies (EBs). The system described herein allows for exploration of how a variety of relevant cell types respond to anticancer treatments. The use of this new organoid model provides a detailed understanding of the cellular response to various drugs, as different cell types vary in their response to anticancer drugs. For example, a regulatory variant that affects gene expression in cardiomyocytes may have a different effect (or none at all) on gene expression in endothelial cells. If this is the case, testing for the effects of these drugs using bulk data, or by using homogenous cell cultures, may result in many false negatives and false positives. In contrast, collecting single-cell data from treated and untreated multilineage cardiovascular organoids described herein provides a unique opportunity to characterize variability in drug-induced CT between multiple individuals and among disease-relevant cell types.

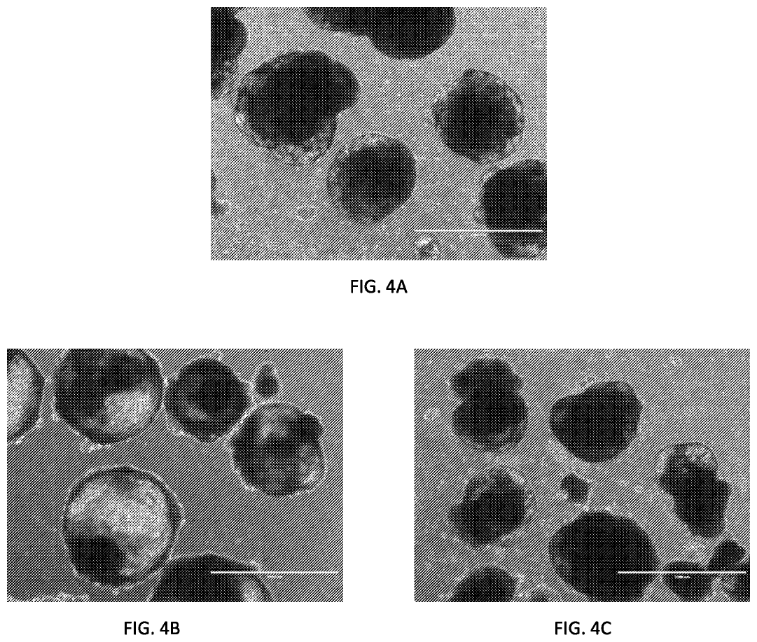

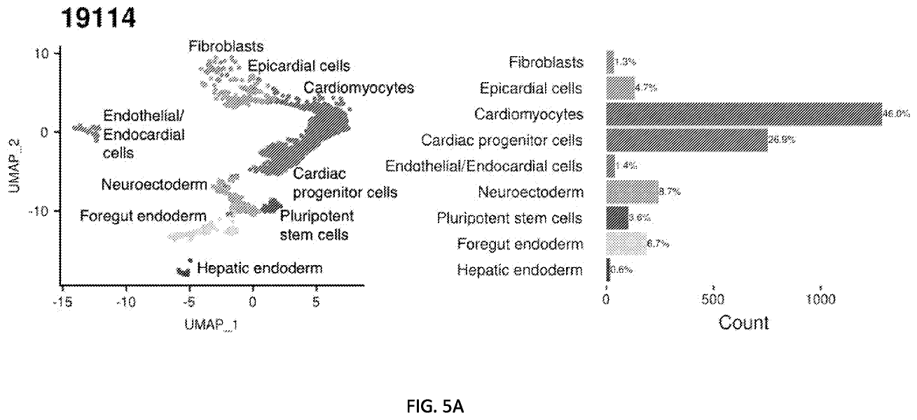

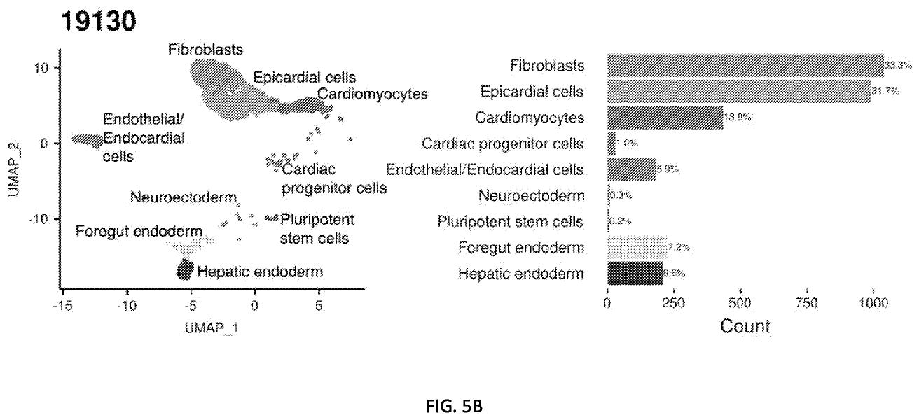

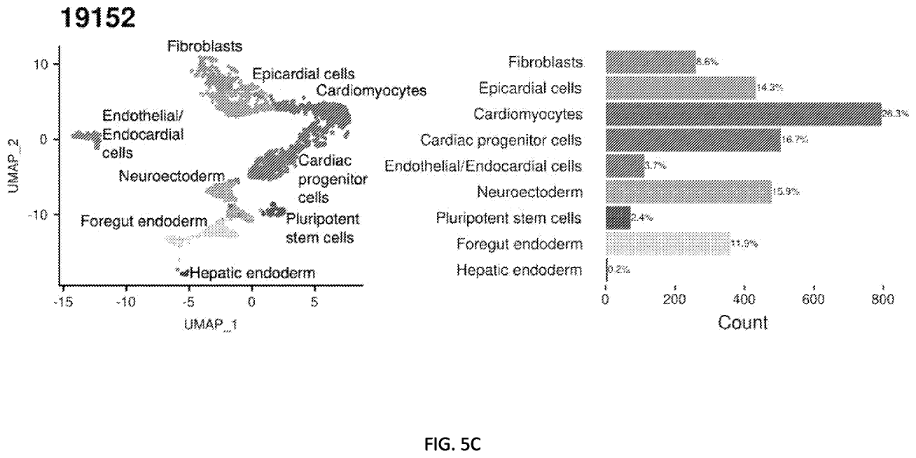

Herein, guided differentiation (FIG. 1A) was performed using three iPSC lines to generate three-dimensional cardiac cell cultures. First, iPSCs were formed into three-dimensional aggregates measuring ˜300 μM in diameter. iPSC aggregates were cultured in the formation plate using Essential 8™ medium (E8) with 10 μM CEPT for 24 hours and then aggregates were transferred to ultra-low attachment plates for an additional 48 hours. iPSC aggregates were then biased towards cardiac lineage using temporal Wnt modulation. Specifically, E8 medium was exchanged for heart medium (RPMI-1640 with a 2% v/v concentration of B-27 supplement, sans insulin) plus the Wnt activator CHIR99021 (“Chiron”) at a final concentration of 4 μM to initiate cardiac differentiation. The heart medium containing CHIR99021 is an exemplary cardiac induction medium as described herein. After 24 hours, the cardiac induction medium (heart medium plus CHIR99021) was exchanged for basal heart medium. The basal heart medium was refreshed 48 hours later, this time adding IWP-4, a Wnt inhibitor, at a final concentration of 2 μM. After 48 hours, IWP-4+ heart medium was replaced with basal heart medium for 48 hours before harvest. Representative images of day 10 aggregates prior to collection are shown in FIGS. 4A, 4B, and 4C.

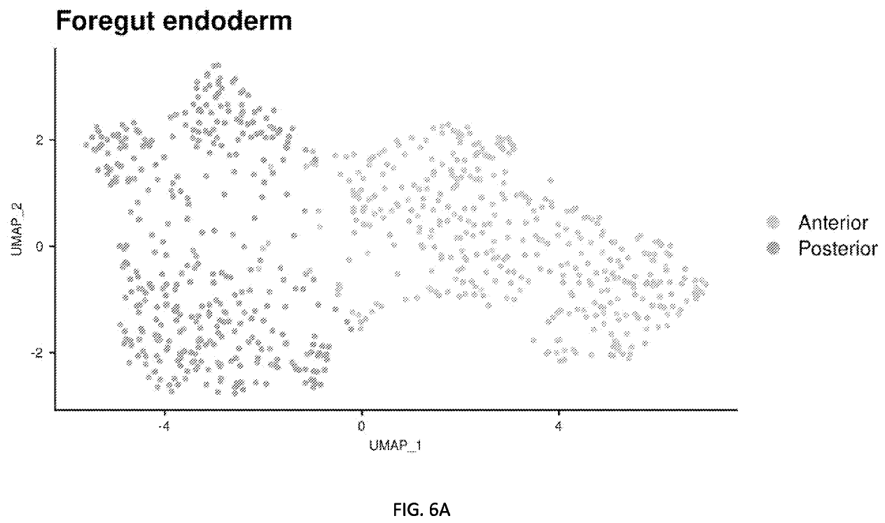

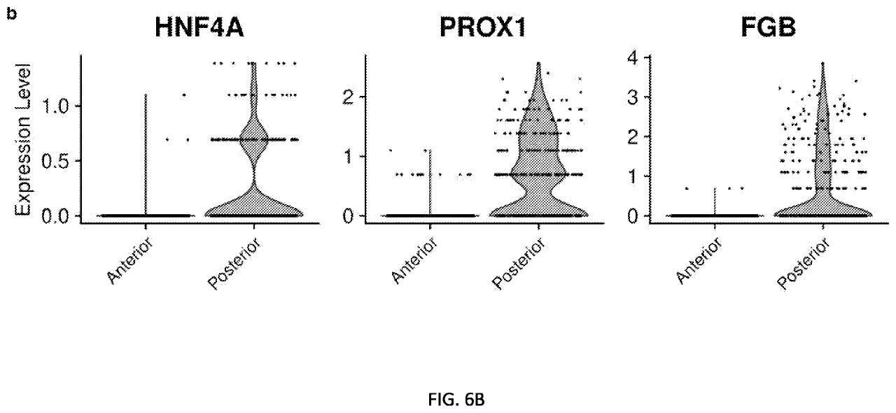

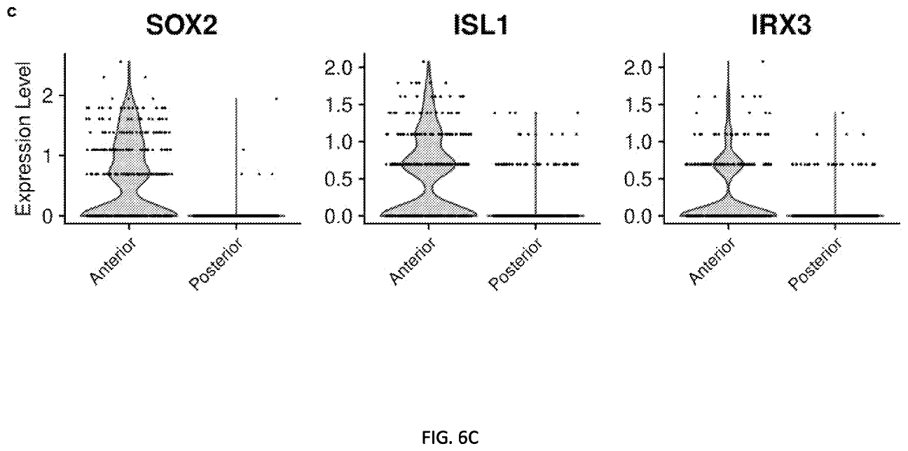

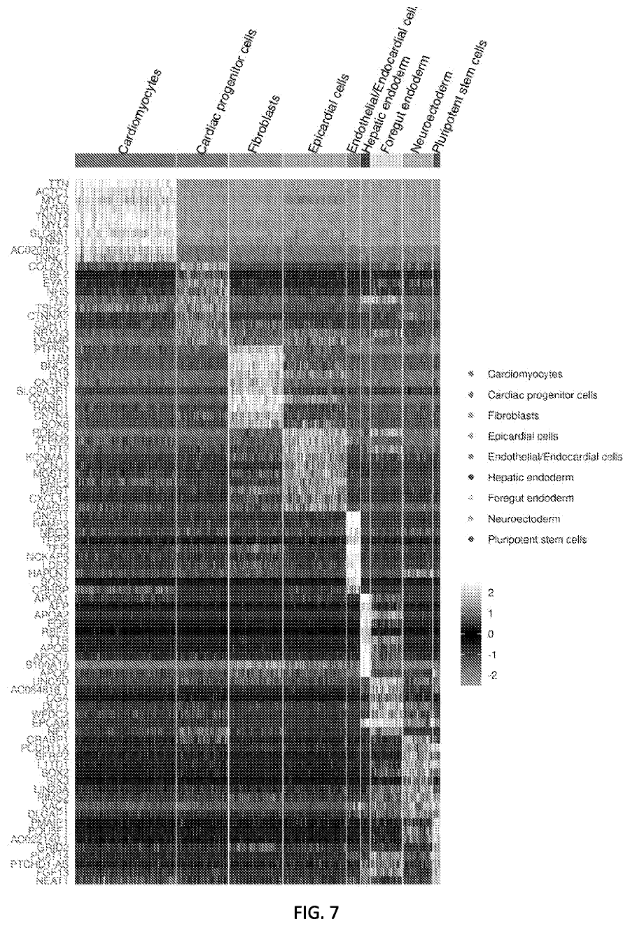

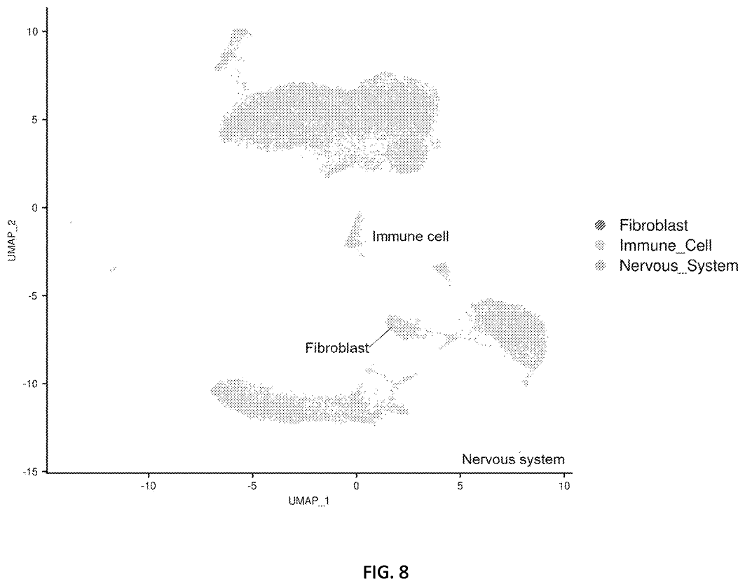

Cells were collected at day 10 using the 10X Genomics platform for sequencing on an Illumina NovaSeq 6000. After filtering and normalizing the data, principal component analysis was performed with 5,000 highly variable features and the top 50 principal components were used for graph-based unsupervised clustering. Guided differentiation cultures included a diversity of cell types from all three germ layers. Cells were annotated using marker gene expression and differential expression analysis (FIG. 1B). Neuroectoderm cells, endothelial/endocardial cells, epicardial cells, fibroblasts, cardiac progenitor cells, and cardiomyocytes were all identified. Cellular populations of foregut endoderm and hepatic endoderm per marker gene expression were annotated (FIG. 1C). A heatmap of the top 10 differentially expressed genes per cluster is shown in FIG. 7. Subcluster analysis of foregut endoderm revealed posterior and anterior foregut populations (FIG. 6).

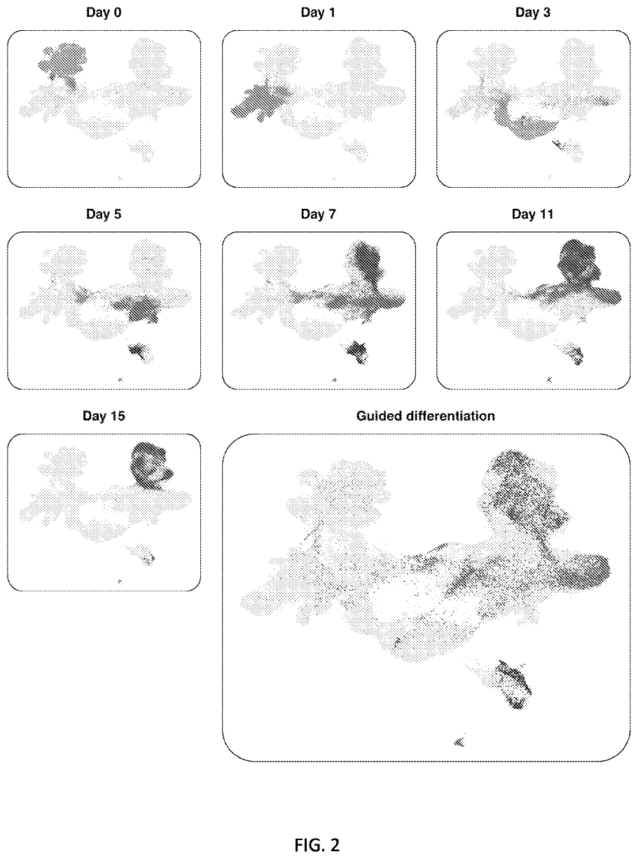

A Seurat integration (Stuart, T. et al. Cell 177, 1888-1902.e21 (2019)) of scRNA-seq data with data from a previous 16-day time-course study of iPSC-CM differentiation performed by Elorbany et al. (Elorbany, R. et al. Single-cell sequencing reveals lineage-specific dynamic genetic regulation of gene expression during human cardiomyocyte differentiation. PLOS Genet. 18, e1009666 (2022)) was performed. Sub-setting cells by the collection day of Elorbany et al. illustrates progression through iPSC-CM differentiation. While cell states were rather specific to certain days in the time-course study, the ‘single time-point’ guided differentiation culture harbored cells throughout the entire UMAP space (FIG. 2), indicating that guided differentiation produced cells that are transcriptionally similar to those collected during the entire time-course study.

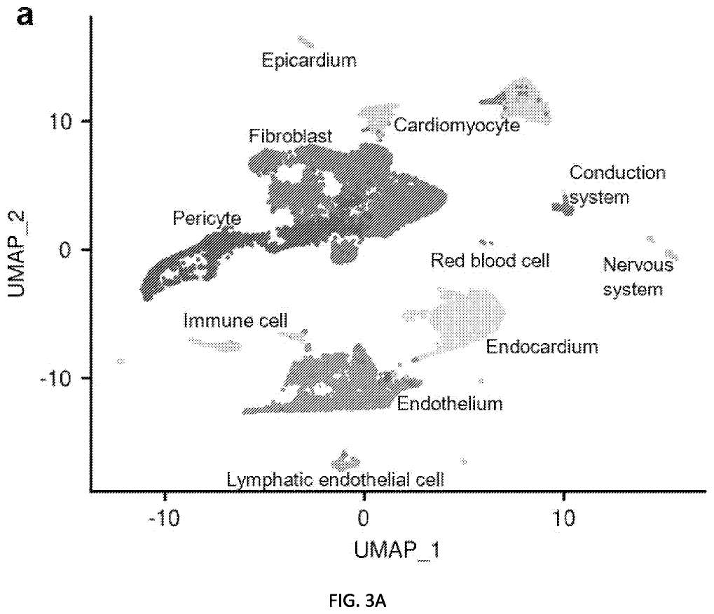

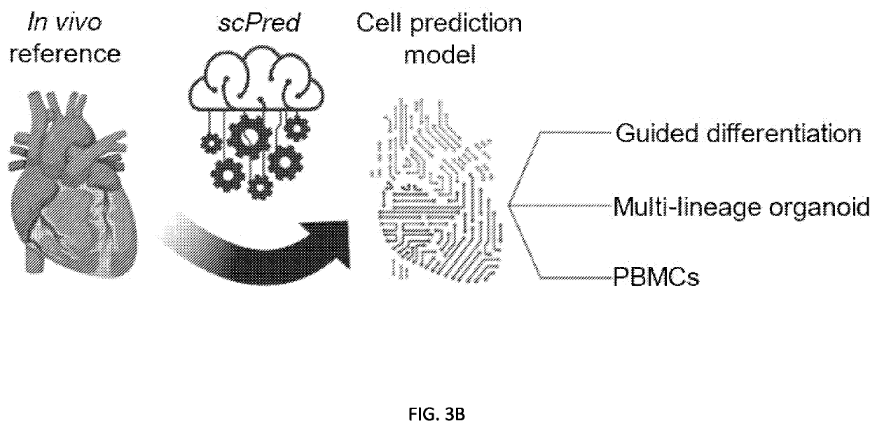

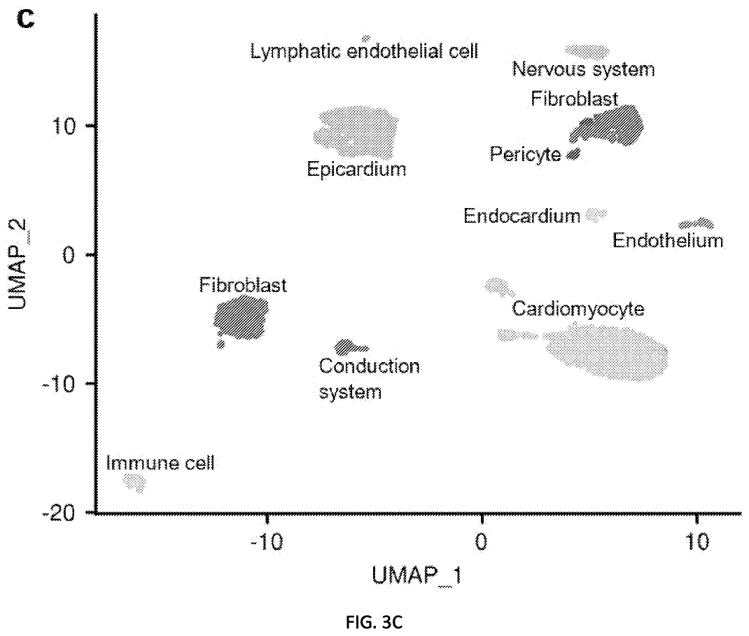

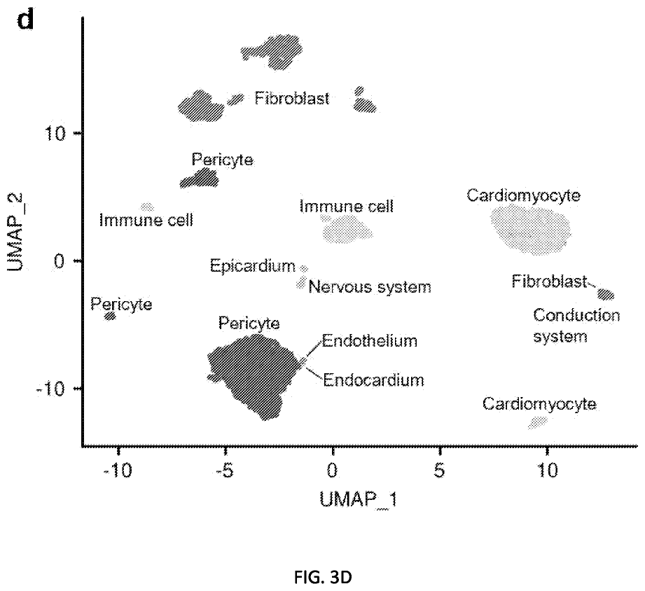

To demonstrate similarity between the transcriptional profiles of cells from guided cultures and their in vivo counterparts, automated cell classification was used. For a cell reference, a dataset of annotated fetal heart cells published by Miao et al. (Miao, Y. et al. Cell Stem Cell 7, 574-589.e8 (2020) was used, which consisted of 11 different cell types (FIG. 3A). This reference was used to train the scPred prediction model (Alquicira-Hernandez, J., Sathe, A., Ji, H. P., Nguyen, Q. & Powell, J. E. Genome Biol. 20, 264 (2019) for automated cell annotation (FIG. 3B). Individual cells from guided differentiation culture were assigned probabilities of belonging to each of the 11 reference cell types. As the guided differentiation culture harbors cell types that are not present in fetal heart tissue (e.g., foregut endoderm), the probability threshold was set to 0.9; cells below the threshold were classified as unassigned and not included in the UMAP plots. Based on this analysis, guided differentiation culture putatively harbored all cell types except red blood cells (FIG. 3C). These included cardiomyocytes, fibroblasts, epicardial cells, endothelial / endocardial cells, and nervous system cells, consistent with the manual cell annotation. Additionally, scPred cell type predictions included immune cells, conduction system cells, and pericytes, which were not detected by manual annotation. ScPred classifies cells individually, without clustering, and is capable of identifying small cell populations that would otherwise be masked by clustering.

Finally, to benchmark guided differentiation culture against established in vitro models, the scPred prediction model was applied to scRNA-seq data from mature multi-lineage organoids (100-day culture) published by Silva, et al. (Silva, A. C. et al. Cell Stem Cell 28, 2137-2152.e6 (2021)) (FIG. 3D). Both iPSC-based cultures showed comparable levels of cell diversity and transcriptional similarity relative to the fetal heart tissue, demonstrating that the guided differentiation culture method can generate diverse, transcriptionally relevant cell types in just 10 days. The reduced time and labor needed for guided differentiation (relative to standard organoids) enables dynamic population-level studies at scale, including eQTL mapping studies at cell type resolution. Moreover, the transcriptional similarity between guided cardiac cell types and their in vivo counterparts indicates that dynamic functional patterns identified using guided cultures are functionally relevant to human tissues.

### Methods

### Samples.

Three iPSC lines were used from unrelated Yoruba individuals from Ibadan, Nigeria (YRI): 19114 (female), 19130 (male), and 19152 (female). These iPSC lines were reprogrammed from lymphoblastoid cell lines (LCLs). Cell line identities were confirmed using genotype data generated by the HapMap project from the original LCL lines (The International HapMap Consortium. Nature 426, 789-796 (2003)).

### IPSC Maintenance.

The iPSC lines were maintained using Matrigel Growth Factor Reduced (GFR) Basement Membrane Matrix (Corning, 354230) with Essential 8™ (E8) medium (Thermo Fisher Scientific, A1517001) and Penicillin-Streptomycin (Lonza, 17-602F) in an incubator at 37° C. and 5% CO₂. At roughly 80% confluency (approximately every 3-5 days), cell cultures were passaged using a dissociation reagent (0.5 mM EDTA, 300 mM NaCl in PBS) and seeded iPSCs with 10 μM ROCK inhibitor Y-27632 (ab120129, Abcam).

### Guided Differentiation.

Similar to directed differentiation, guided differentiation primarily generates cardiac-relevant cell types. However, it also yields cell types representative of all three germ layers and includes cell stages throughout cardiomyogenic lineage, resulting in a greater diversity of cell types. Guided differentiation of the 3 iPSC lines was performed by forming three-dimensional aggregates and performing temporal Wnt modulation. iPSC aggregates were formed using an AggreWell™800 24-well plate (STEMCELL Technologies, 34811). Each well was coated with Anti-Adherence Rinsing Solution (STEMCELL Technologies, 07010). On day 0, iPSCs were disassociated and seeded onto the plate using E8 medium with 10 μM CEPT, i.e., chroman 1 (Torcis, 7163), emricasan (MedKoo Biosciences, 510230), polyamines (Sigma-Aldrich, P8483), and trans-ISRIB (Torcis, 5284). Cell aggregates were formed at approximately 300 μm diameter using 2 million cells per well (i.e., 1 million cells per mL) of the Aggre Well™800 plate. The plate was centrifuged at 100 g for 3 min to aggregate cells together. Cells remained in the plate for 24 h. After 24 h, fully-formed iPSC aggregates were transferred to a suspension culture using an ultra-low adherent plate (STEMCELL Technologies, 100-0083) with E8 medium for 48 h. On day 3, the cell medium was changed to heart medium plus 4 μM CHIR99021 (STEMCELL Technologies, 72052). Heart medium is comprised of RPMI 1640 Medium, GlutaMAX™ Supplement, HEPES (Thermo Fisher Scientific, 72400047), 2% v/v B-27 Supplement, minus insulin (Thermo Fisher Scientific, A1895601), and Penicillin-Streptomycin. The heart medium with CHIR99021 is referred to herein as the cardiac induction medium. After 24 h, on day 4, the cardiac induction medium (heart medium 30 CHIR99021) was changed to basal heart medium for 48 h. On day 6, medium was changed to heart medium plus 2 μM IWP-4 (STEMCELL Technologies, 72552) for 48 h. Lastly, on day 8, medium was changed to basal heart medium for 48 h and cells were collected on day 10. Contraction became visible on day 8, 5 days after Wnt activation. Organoids experienced both Wnt activation and Wnt inhibition before contraction was observed.

### Aggregate Disassociation.

Day 10 aggregates were collected from the 3 iPSC lines and disassociated by treating with room temperature AccuMax™ (STEMCELL Technologies, 07921) followed by incubation at 37° C. for 10 min. Following incubation, aggregates were pipetted up-and-down for 30 sec with a p 1000 wide bore pipette tip (Thermo Fisher Scientific, 2079GPK). Aggregates were then incubated for an additional 5 min at 37° C. Pipetting was repeated every 5 minutes until aggregates were disassociated, at which point 5 mL of 4° C. Bovine Serum Albumin (BSA) solution was added (Sigma-Aldrich, A8412) and cells were centrifuged for 100 g for 3 min. Cells were resuspended in 1 mL 4° C. BSA solution and strained through a 40 μm cell strainer (Bel-Art™, H136800040). Cells were combined from each iPSC line in even proportions (500,000 cells per line) and centrifuged at 100 g for 3 min. Cells were resuspended in 4° C. BSA solution at a concentration of approximately 2 million cells per mL. Finally, the cell suspension was strained using a 40 μm cell strainer.

### ScRNA-Seq Analysis.

Cells were collected from the 3 iPSC lines for scRNA-seq using the 10X Genomics Chromium Next GEM Single Cell 3′ Reagent Kits v3.1 (Dual Index). 10,000 cells total were targeted in one lane of a 10X chip and the library was sequenced using an Illumina NovaSeq 6000 at the University of Chicago Functional Genomics Core Facility. 20,755 mean reads per cell were obtained. Samples were aligned to the human genome (GRCh38) using Cell Ranger v6.1.2 and cells were assigned to individuals using Vireo v0.5.6. Count data was analyzed in R (v4.2.0)/ RStudio (v2022.02.3+492) using Seurat v4.3.0 with tidyverse (v1.3.1.

For cell filtering, cells classified as doublets or unassigned were removed by Vireo. Additionally, cells expressing less than 1,500 unique genes were filtered out. Sctransform-based normalization (v0.3.5) (sctransform function) was performed using 5,000 variable features, dimensionality reduction (RunPCA function) was performed, and 50 dimensions were used for uniform manifold approximation and projection (UMAP) embedding (RunUMAP function). Nearest neighbors were computerized using 50 dimensions (FindNeighbors function) and unsupervised clustering was performed at a resolution of 0.2 (FindClusters function) to yield 9 cell clusters for differential expression analysis using the Wilcoxon Rank Sum test. Cell clusters were annotated based on canonical marker gene expression. Cell clusters were shaded using color schemes for all figures unless otherwise noted.

### Foregut Endoderm Subcluster Analysis.

The foregut endoderm cluster cells were subset as their own Seurat object and re-normalized using the same approach described above. Unsupervised clustering was performed at a resolution of 0.1 to yield 2 clusters for differential expression analysis. Cell clusters were annotated based on canonical marker gene expression.

### Seurat Integration.

In order to compare cells from guided differentiation and cells from a 16-day iPSC-CM differentiation time-course published by Elorbany et al. (2022), Seurat integration of the two scRNA-seq datasets was performed. First, the iPSC-CM time-course dataset was filtered using the same parameters as in the original study; that is, 1) genes must be detected in at least 10 cells, 2) cells must contain at least 300 unique genes, 3) cells must have no more than 25% mitochondrial reads, 4) cells must have a doublet probability of 0.3 or less, 5) cell assignment must be unambiguous, 6) cells with feature or read counts more than 4 standard deviations away from the median are excluded. Cells were filtered from the guided differentiation dataset using the criteria described in the scRNA-seq analysis section above. Using sctransform, each dataset was normalized individually using 5,000 variable features and 5,000 anchor features were selected for integration (SelectIntegrationFeatures function). The datasets were prepared for integration (PrepSCTIntegration function), a set of anchor features was determined (FindIntegrationAnchors function), and the datasets were integrated (IntegrateData function). Following integration, dimensionality reduction, UMAP embedding, and computation of nearest neighbors were performed as described above. Unsupervised clustering was performed at a resolution of 0.1 to yield 7 cell clusters. Cells were subset by dataset (and for the time-course dataset, by day of collection) for visualization with UMAP.

### Automated Cell Classification.

scPred v1.9.2 was used to create a prediction classifier model trained on annotated fetal heart cells published by Miao et al. (2020). The fetal heart dataset comprised 11 annotated cell types: endocardium, endothelium, lymphatic endothelial cell, cardiomyocyte, epicardium, smooth muscle cell, fibroblast, immune cell, nervous system, conduction system, and red blood cell. The fetal heart dataset was normalized using the same approach described above and the classifier (getFeatureSpace and trainModel functions) was trained using default parameters. For the multi-lineage organoid dataset, cells expressing less than 1,500 unique genes were filtered out. The multi-lineage organoid dataset, guided differentiation dataset, and peripheral blood mononuclear cells (PBMC) dataset (20 k Human PBMCs, 3′ HT v3.1, Chromium X Single Cell Gene Expression Dataset by Cell Ranger (2021)) were all normalized using the same approach described above. Cells from each dataset (scPredict function) were classified using a threshold of 0.9. Cell clusters in FIG. 3 were shaded using RColorBrewer (v1.1-3).

### Exemplary Marker Genes

The following represent exemplary marker genes that can be used for annotation of different cell types.

### Cardiac Cells.

Cardiac progenitor cells can be annotated based on expression of the canonical markers ISL1, NKX2-5, and PDGFRA; and proliferating cells can be identified by expression of cell cycle genes MKI67 and UBE2C. Cardiomyocytes can be classified using expression of sarcomere genes (TNNT2 and ACTN2) and ion channel genes (SLC8A1 and KCNH7). Within the cardiomyocyte cluster, marker gene expression can be observed for mature cardiomyocytes (TNNI3 and MYH7) as well as for cardiomyocyte subtypes, including ventricular (FHL2 and IRX4), atrial (MYL7 and NPPA) and nodal (HCN1 and HCN4) cardiomyocytes. Finally, expression of smooth muscle cell markers (ACTA2, TAGLN, and CNNI) within the cardiomyocyte cluster can also be observed.

### Fibroblasts.

Fibroblasts and myofibroblasts can be identified using expression of COL3A1 as well as the canonical fibroblast markers PDGFRA and TCF21. Myofibroblasts can be distinguished from fibroblasts by expression of ACTA2, DCN, and LUM. Additionally, myofibroblasts may show reduced expression of PDGFRA and TCF21 but increased expression of COL3A1 relative to fibroblasts.

### Endoderm and Ectoderm.

Two endoderm lineage clusters may be identified: an endoderm epithelium cluster marked by expression of EPCAM and CDH1, and a foregut cluster containing liver cells (AFP and SERPINA1) and intestinal cells (APOB). Endoderm may support cardiomyocyte differentiation. An ectoderm cluster can be identified using PAX6 expression, and neural crest cells can be classified using PAX3 expression.

In some embodiments, the methods described herein involve annotating/identifying cells. Annotated cells (e.g. identified cells) can then be analyzed. For instance, a response such as an eQTL to the agent or the condition in at least one identified/annotated cell type can be evaluated. One commonly employed approach to cell annotation (e.g. cell type identification) utilizes unsupervised clustering algorithm to group cells together into ‘clusters’ based upon each cell's transcriptomic profile. Genes that are differentially expressed in one cluster of cells relative to all other clusters (at a level that reaches statistical significance) can potentially be used to classify that cluster of cells, assuming the expression is specific to a particular cell type. As an example, PAX6 is a marker for neuroectoderm differentiation; therefore, a cluster of cells that differentially expresses PAX6 could be classified as neural progenitor cells. In addition, reference datasets can also be used wherein the unclassified cells are matched with reference transcriptomic profiles from known cell types (e.g., cells biopsied from a human heart).

Cells often do not fit neatly into discrete categories, and the concept of ‘cell type’ may be considered a continuous variable. Accordingly, in some embodiments a non-negative matrix factorization (NMF) can be used to approximate the composition of cell types and cell states that comprise a single cell. In other words, the gene expression of a single cell may partially match with several different cell types, and NMF is used to estimate cell type coefficients. The approach or combination of approaches used for cell classification is generally dataset-specific.

### Example 2

Most studies of CT have focused on anthracyclines such as doxorubicin (DOX). However, other types of anticancer drugs are also toxic to the heart and vascular system. Moreover, drugs for diseases other than cancer can exhibit cardiotoxicity, and can be evaluated using the multilineage cardiovascular organoid systems described herein. Although anthracyclines are notorious for their cardiotoxic effects, they are far from unique. Antimetabolites, alkylating agents, and other types of drugs also induce CT. Even targeted therapies, which were initially thought to be safer than non-targeted treatments, have been associated with a range of deleterious cardiovascular effects. Accordingly, an investigation of cardiotoxicity of various agents, including anticancer agents such as anthracyclines, alkylating agents, anti-metabolites, and VEGF-inhibitors, is needed. The multilineage cardiovascular organoids provided herein address this need by providing a platform for investigation of cardiotoxicity using any desired agent.

Multilineage cardiovascular organoids can be developed as described in Example 1. Single-cell expression data can be collected from control and drug-treated samples to map eQTLS. Any suitable type of single-cell expression data can be collected and used, including, for example, single-cell sequencing (e.g. single-ATAC-seq, described in Nat Methods 2013 Dec; 10(12): 1213-8)

### Selection of Drugs.

The multilineage cardiovascular organoid can be treated with any desired agent. In some embodiments, the agent can be an anticancer drug. Various anticancer drugs can be tested, including but not limited to DOX, the anti-metabolite 5-fluorouracil (5-FU), and the VEGF inhibitor bevacizumab (BVC), both of which are associated with CT. Mapping response eQTLs for three different drugs for exploration of whether DOX, 5-FU, and BVC induce cardiovascular dysfunction through distinct or shared pathways and cell types. In some embodiments, drugs with unknown cardiac effect can be tested, such as to determine whether a given agent/drug induces cardiotoxicity. For example, an anticancer agent with unknown cardiac effect can be tested on the multilineage cardiovascular organoid described herein to evaluate cardiotoxicity of the agent.

### Drug Treatment.

Cardiac embryoids can be treated with any suitable dose of the desired agent (e.g. anticancer agent). For example, an appropriate dose delivered to the cardiac embryoid may be substantially equivalent to a therapeutic dose that would be delivered to a human subject. In some embodiments, the dose can be 0.625, 1.25, 2.5, or 5 μM for about 24 hours. Cell viability can be assessed after 24 hours scRNA-seq data collected from treated cells can be used to determine the optimum dose.

### Dose Optimization.

To find the appropriate dose for in vitro testing of drug response, a multilineage cardiovascular organoid can first be treated with a range of concentrations of a particular drug and a variety of parameters can be assessed. Suitable parameters include, for example, cell death and gene expression. For example, overall cell death can be assessed using microscopy, live/dead cell staining, and/or cell counting. Cell death in particular cell types can be assessed using flow cytometry, qPCR, and/or single cell sequencing. If only certain cell types die in response to drug treatment, this is indicative of cell-type specific drug toxicity. Expression of genes associated with cell stress and apoptosis can be evaluated, such as by using low-depth single cell sequencing and qPCR. On-target therapeutic effects on gene expression (when the mechanism of action of the drug is well characterized) can be assessed using low-depth single cell sequencing and qPCR.

The ideal dose for in vitro testing should cause limited cell death and stress response across all cell types and will show evidence of on-target effects. Additionally, the ideal dose should show similar effects across at least 3 individuals and 3 replicates of testing.

Results from dose-range studies in the multilineage cardiovascular organoids can inform dosing and toxicity testing in vivo. If cell-type specific toxicity arises at particular doses, animal studies should include specific testing of the affected cell types or tissues. For example, if treatment results in specific toxicity to retinal cells, both animal testing and tests in human trials should incorporate additional assays, measurements, and/or endpoints to assess retinal health.

### Exemplary Study Design.

Multilineage cardiovascular organoids can be grown as described in Example 1, with two replicates per individual per condition. On a suitable day of organoid differentiation, multilineage cardiovascular organoids can be exposed to the desired agent or a negative control (vehicle only) for a suitable duration of time (e.g. 24 hours) followed by a media change and dissociation in preparation for scRNA-seq. For example, in some embodiments day 8 is a suitable day of organoid differentiation at which the multilineage cardiovascular organoids can be exposed to the desired agent. However, other days may be used (e.g. day 7, day 8, day 9, day 10, or other days).

### Data Collection and Sequencing.

Mechanical dissociation techniques can be used to collect cells for sequencing. Sequencing can be performed on the 10X Genomics platform. In some methods, at least about 5,000 cells from each well with at least about 20-30,000 reads sequenced per cell, can be used as parameters for sequencing. The RNA can be sequenced on an Illumina NovaSeq 6000. Sequencing reads can be used to confirm the genotype of each cell line and therefore authenticate it. This authentication analysis can be used as a standard quality control measure.

### Data Preprocessing.

All mRNA fragments may be tagged with a cell-specific barcode and UMI. Reads can be aligned to the human genome (GRCh38) and each aligned read assigned to a genomic feature to create a count matrix representing the frequency of each feature in the data set. Undesirable cells can be excluded. For example, cells with data from fewer than 1,500 genes and cells with a doublet probability greater than 30% can be excluded.

Identification of Genetic Variants that Regulate the Transcriptomic Response of Multilineage Cardiovascular Organoids to Each Drug.

Using existing genome-wide genotype data from the iPSC panel and the single-cell gene expression data that can be obtained using the methods described herein, response eQTLs for agents can be mapped. Because multilineage cardiovascular organoids consist of multiple different cell types at various stages of differentiation, cells can be first assigned to clusters and classified into cell types, such as by the methods described in Example 1. In addition to mapping response eQTLs for each drug treatment, steady state eQTLs in untreated cells can also be mapped. Use of single-cell data will allow for distinguishing between true eQTLs affecting a specific cell type and regulatory differences driven by potentially non-genetic differences, such as changes in cell type composition.

Mapping Response eQTLs (Effect Size Changes with Treatment).

Response eQTLs can be mapped in multilineage cardiovascular organoids by extending the method developed to map dynamic eQTLs from bulk RNA-seq data (Strober, B. J. et al. Dynamic genetic regulation of gene expression during cellular differentiation. 5 (2019 )). Cis eQTLs can be focused on, as the majority of convincing associations lie within 100 kb of the target gene and because a much larger sample size is needed to effectively map trans eQTLs. After filtering data for lowly-expressed genes, cis eQTLs can be mapped independently for each cell type and exposure condition using pseudobulk gene expression data. A linear regression model can be used to examine associations between variant genotypes and expression levels. Age, sex, collection batch, dissociation time and other measured factors with potential biological and technical effects can be accounted for by including them as covariates in the model. Unmeasured surrogate confounders can also be accounted for by performing PCA on a correlation matrix of gene expression values for each condition and cell type. An empirical determination of how many PCs should be regressed out of the data in order to detect the largest number of eQTLs in each condition and cell type can be performed. To account for multiple hypothesis testing, multiple permutations of genotypes (e.g. 1,000 permutations of the genotypes) can be developed, and this empirical null distribution can be used to adjust eQTL p-values. Storey's procedure to calculate FDRs can be applied, and genes with significant eQTLs will be reported at an FDR<10%.

### Potential Alternative Strategies.

Response eQTLs can be mapped by considering the difference in gene expression levels between the control and treated condition as the mapped phenotype. Loci in which genetic variation is correlated with inter-individual variation in the difference in gene expression levels between the control and treated conditions are, by definition, response eQTLs. As an alternative approach, for a given cell type, a response eQTL can be defined as either (i) significant only in the control condition, (ii) significant in any of the treatment conditions but not in the control condition, or (iii) significant in both conditions, but with different effect sizes. To classify these, the effect size of an eQTL can be tested as to whether it is significantly different between two conditions using a standard z-test. If, for example, there is a significant eQTL only in the treatment condition, the estimated effect sizes of the significant treatment eQTL and the non-significant association in the control condition (which, as it was classified as ‘not significant’, must overlap zero by definition), can be required to not overlap within 2 s.d. of the respective means. The resulting z-score based p-values will be corrected for multiple testing using Bonferroni correction (p_{beta}<0.05).

In some embodiments, additional analyses can be employed to analyze the multilineage cardiovascular organoids. Generally speaking, any suitable analysis can be applied in order to evaluate the response of one or more cell types within the multilineage cardiovascular organoids to an agent or condition. For example, differential expression analysis between treatment and control groups can be applied to each cell type. As another example, differentially expressed genes and/or pathways can be characterized in order to evaluate the response of one or more cell types to the agent or condition. As another example, genes with increased expression can be determined following exposure to the agent or condition, which lends an understanding to genes associated with cell stress and/or toxicity.

Multiple approaches can be leveraged to contextualize differentially expressed genes (DEGs) and associated response eQTLs (reQTLs). For instance, DEGs can be explored through Gene Ontology (GO) analysis to determine each gene's biological properties (function, pathway, location, etc.). In addition, cardiac organoid data can be compared with other in vitro studies using similar treatments and models (e.g., cardiac organoids or pluripotent stem cell-derived cardiomyocytes). Further, these can be compared with data from relevant genome-wide association studies (GWAS) to identify overlap of variants between the datasets; overlap may indicate a role in disease or injury.

Genetic variants that regulate the transcriptional response to a drug can provide insight into cardiovascular toxicity (CT) risk, particularly when those variants are assayed in relevant cell types. Some of the earliest response eQTL experiments examined drug responses in vitro, treating cell lines with steroids, statins, and a range of other disease-relevant chemicals. By comparing response eQTLs with variants already identified in GTEx (GTEx Consortium. The GTEx Consortium atlas of genetic regulatory effects across human tissues. Science 369, 1318-1330 (2020) and in GWAS, these studies give insight into gene-environment interactions that may play a causal role in drug responses and disease. The methods described herein can comprise comparing response eQTLs as measured herein with variants identified in GTEx and/or GWAS to give insight into gene-environmental interactions that may play a causal role in drug response (e.g. cardiac toxicity).

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A multilineage cardiovascular organoid comprising mesodermal and non-mesodermal cell types.

2. The multilineage cardiovascular organoid of claim 1, wherein the mesodermal cell types comprise one or more cell types selected from cardiac progenitor cells, proliferating cells, cardiomyocytes, and fibroblasts.

3. The multilineage cardiovascular organoid of claim 2, wherein the mesodermal cell types comprise 3 or more cell types.

4. The multilineage cardiovascular organoid of claim 3, wherein the mesodermal cell types comprise cardiac progenitor cells, proliferating cells, cardiomyocytes, and fibroblasts.

5. The multilineage cardiovascular organoid of any one of the preceding claims, wherein the non-mesodermal cell types comprise endoderm epithelial cells, liver cells, intestinal cells, and/or cardiac neural crest cells.

6. The multilineage cardiovascular organoid of any one of the preceding claims, wherein the mesodermal cell types comprise cardiac progenitor cells, proliferating cells, cardiomyocytes, and fibroblasts, and wherein the non-mesodermal cell types comprise endoderm epithelial cells, liver cells, intestinal cells, and cardiac neural crest cells.

7. The multilineage cardiovascular organoid of any one of the preceding claims, wherein the mesodermal and non-mesodermal cell types are determined by expression of one or more markers for each cell type.

8. The multilineage cardiovascular organoid of any one of the preceding claims, for use in a method of assessing cardiotoxicity of an agent or condition.

9. The multilineage cardiovascular organoid of claim 8, wherein the method of assessing cardiotoxicity comprises contacting the multilineage cardiovascular organoid with the agent or subjecting the multilineage cardiovascular organoid to the condition, and evaluating a response to the agent or the condition in at least one cell type within the multilineage cardiovascular organoid.

10. The multilineage cardiovascular organoid of claim 9, wherein evaluating a response to the agent or the condition in the at least one cell type within the multilineage cardiovascular organoid comprises identifying one or more response expression quantitative loci (eQTLs) in the at least one cell type.

11. The multilineage cardiovascular organoid of claim 9, wherein the one or more response eQTLs are identified using single-cell RNA sequencing (scRNA-seq).

12. A method of generating a multilineage cardiovascular organoid, the method comprising:
a. culturing an embryoid body in a cardiac induction medium comprising a Wnt signaling activator; and
b. culturing the embryoid body in a Wnt inhibitor medium comprising a Wnt signaling inhibitor.

13. The method of claim 12, further comprising culturing the embryoid body in a basal heart medium in between culturing the embryoid body in the cardiac induction medium and culturing the embryoid body in the Wnt inhibitor medium.

14. The method of claim 12 or claim 13, wherein the Wnt signaling activator is a small molecule GSK3 inhibitor.

15. The method of claim 14, wherein the small molecule GSK3 inhibitor is CHIR99021.

16. The method of claim 15, wherein the cardiac induction medium comprises CHIR99021 at a concentration of about 4 μM.

17. The method of any one of claims 12-15, wherein the Wnt signaling inhibitor is a small molecule porcupine inhibitor.

18. The method of claim 17, wherein the small molecule porcupine inhibitor is IWP-4, IWP-2 LGK974, C59, or ETC-159.

19. The method of claim 18, wherein the Wnt inhibitor medium comprises IWP-4 at a concentration of about 2 μM.

20. The method of any one of claims 12-19, wherein the embryoid body is generated from induced pluripotent stem cells (iPSCs).

21. A method of generating a multilineage cardiovascular organoid, comprising:
a. generating an embryoid body from induced pluripotent stem cells (iPSCs); and
b. guiding differentiation of the embryoid body to a cardiac lineage, wherein guiding differentiation of the embryoid body to a cardiac lineage comprises culturing an embryoid body in a cardiac induction medium comprising a Wnt signaling activator.

22. The method of claim 21, wherein guiding differentiation further comprises culturing the embryoid body in a Wnt inhibitor medium comprising a Wnt signaling inhibitor following culturing the embryoid body in the cardiac induction medium.

23. The method of claim 22, wherein guiding differentiation further comprises culturing the embryoid body in a basal heart medium in between culturing the embryoid body in the cardiac induction medium and culturing the embryoid body in the Wnt inhibitor medium.

24. The method of any one of claims 21-23, wherein the Wnt signaling activator is a small molecule GSK3 inhibitor.

25. The method of claim 24, wherein the small molecule GSK 3 inhibitor is CHIR99021.

26. The method of claim 25, wherein the cardiac induction medium comprises CHIR99021 at a concentration of about 4 μM.

27. The method of any one of claims 24-26, wherein the Wnt signaling inhibitor is a small molecule porcupine inhibitor.

28. The method of claim 27, wherein the small molecule porcupine inhibitor is IWP-4, IWP-2, LGK974, C59, or ETC-159.

29. The method of claim 28, wherein the Wnt inhibitor medium comprises IWP-4 at a concentration of about 2 μM.

30. A system comprising a plurality of multilineage cardiovascular organoids, wherein each of the plurality of multilineage cardiovascular organoids comprises mesodermal and non-mesodermal cell types.

31. The system of claim 30, wherein the mesodermal cell types comprise one or more cell types selected from cardiac progenitor cells, proliferating cells, cardiomyocytes, fibroblasts, and myofibroblasts.

32. The system of claim 31, wherein the mesodermal cell types comprise 3 or more cell types.

33. The system of claim 32, wherein the mesodermal cell types comprise cardiac progenitor cells, proliferating cells, cardiomyocytes, and fibroblasts.

34. The system of any one of claims 30-33, wherein the non-mesodermal cell types comprise endoderm epithelial cells, liver cells, intestinal cells, and/or cardiac neural crest cells.

35. The system of any one of claims 30-34, wherein the mesodermal cell types comprise cardiac progenitor cells, proliferating cells, cardiomyocytes, and fibroblasts, and wherein the non-mesodermal cell types comprise endoderm epithelial cells, liver cells, intestinal cells, and cardiac neural crest cells.

36. The system of any one of claims 30-35, wherein the mesodermal and non-mesodermal cell types are determined by expression of one or more markers for each cell type.

37. The system of any one of claims 30-36, wherein the plurality of multilineage cardiovascular organoids are generated by guided differentiation of a plurality of embryoid bodies derived from iPSCs of varying genotypes.

38. The system of any one of claims 30-37, for use in a method of assessing cardiotoxicity of at least one agent or condition.

39. The system of claim 38, wherein the method of assessing cardiotoxicity comprises contacting at least one of the plurality of multilineage cardiovascular organoids with the agent or subjecting at least one of the plurality of multilineage cardiovascular organoids to the condition and evaluating a response to the agent or the condition in at least one cell type within the at least one multilineage cardiovascular organoid.

40. The system of claim 39, wherein evaluating a response to the agent or the condition in the at least one cell type within the at least one multilineage cardiovascular organoid comprises measuring gene expression, activity, or regulation in the at least one cell type.

41. The system of claim 39 or claim 40, wherein evaluating a response to the agent or the condition in the at least one cell type within the at least one multilineage cardiovascular organoid comprises identifying one or more response expression quantitative loci (eQTLs) in the at least one cell type.

42. The system of claim 41, wherein the one or more response eQTLs are identified using single-cell RNA sequencing (scRNA-seq).

43. A method comprising exposing the multilineage cardiovascular organoid of any one of claims 1-11 or the system of any one of claims 30-37 to an agent or condition, and evaluating a response to the agent or the condition in at least one cell type within the multilineage cardiovascular organoid or within at least one multilineage cardiovascular organoid within the system.

44. The method of claim 43, wherein evaluating a response to the agent or the condition in the at least one cell type within the at least one multilineage cardiovascular organoid comprises measuring gene expression, activity, or regulation in the at least one cell type.

45. The method of claim 43 or claim 44, wherein evaluating a response to the agent or the condition in the at least one cell type comprises identifying one or more response expression quantitative loci (eQTLs) in the at least one cell type.

46. The method of claim 45, wherein the one or more response cQTLs are identified using single-cell RNA sequencing (scRNA-seq).

47. A method of assessing cardiotoxicity of an agent or condition, the method comprising exposing the multilineage cardiovascular organoid of any one of claims 1-11 or the system of any one of claims 30-41 to an agent or condition, and evaluating a response to the agent or the condition in at least one cell type within the multilineage cardiovascular organoid or within at least one multilineage cardiovascular organoid within the system.

48. The method of claim 47, wherein evaluating a response to the agent or the condition in the at least one cell type within the at least one multilineage cardiovascular organoid comprises measuring gene expression, activity, or regulation in the at least one cell type.

49. The method of claim 47 or claim 48, wherein evaluating a response to the agent or the condition in the at least one cell type comprises identifying one or more response expression quantitative loci (eQTLs) in the at least one cell type.

50. The method of claim 49, wherein the one or more response eQTLs are identified using single-cell RNA sequencing (scRNA-seq).
